(19) United States
(12) Patent Application Publication
(10) Pub. No.: US 2014/0302068 A1
(43) Pub. Date: Oct. 9, 2014

(54) MicroRNA Biomarkers for Diagnosing Parkinson's Disease

(71) Applicants: Sok Kean Khoo, Ada, MI (US); Aik-Choon Tan, Englewood, CO (US)

(72) Inventors: Sok Kean Khoo, Ada, MI (US); Aik-Choon Tan, Englewood, CO (US)

(73) Assignees: The Regents of the University of Colorado, Danver, CO (US); Van Andel Research Institute, Grand Rapids, MI (US)

(21) Appl. No.: 14/342,345

(22) PCT Filed: Sep. 10, 2012
(86) PCT No.: PCT/US12/54470

§ 371 Date: Jun. 6, 2014

Related U.S. Application Data
(60) Provisional application No. 61/532,718, filed on Sep. 9, 2011.

(51) Int. Cl. C12Q 1/68

(52) U.S. Cl. CPC C12Q 1/6874
USPC 424/184.1; 506/9; 536/24.33

(57) ABSTRACT

The identification, development and validation of plasma-based circulating microRNA (miRNAs) biomarkers useful in determining if a subject has Parkinson's disease (PD), is at increased risk of developing PD, or has PD that is progressing or is in remission are presented.

This application claims priority to U.S. Provisional Application No. 61/532,718 filed on Sep. 9, 2011, which is incorporated herein by reference in its entirety.

### CROSS REFERENCE TO SEQUENCE LISTING

This application incorporates by reference in its entirety the Sequence Listing entitled “314998 SEQUENCE LISTING_ST25.txt” (3.97 kilobytes), which was created Sep. 1, 2011 and is filed electronically herewith.

### BACKGROUND OF THE INVENTION

Parkinson's disease (PD) is the second most common neurodegenerative disorder, affecting approximately 1 million Americans and 5 million people worldwide. Its prevalence is projected to double by 2030. Definite diagnosis for PD can only be made postmortem, for instance, by the characteristic accumulation of the protein alpha-synuclein into Lewy body inclusions in neurons. Usually, clinical diagnoses of PD are based on characteristic motor findings, which may include resting tremor, rigidity, bradykinesia, and postural instability. Currently, the diagnosis of PD is based on fitting observed symptoms and their severity into clinical rating scales such as the Unified Parkinson's Disease Rating Scale (UPDRS) or UK Parkinson's Disease Society Brain Bank Research Center (UKPDSBRC) clinical diagnostic criteria. Clinical assessments are subjective, however; the accuracy of diagnosis for PD is not 100% even by experienced movement disorder specialists. Further, by the time of diagnosis, an estimated 60-70% of the patient's dopaminergic neurons have already been lost. Diagnosing PD at its early stages can be difficult, especially when essential tremor and atypical Parkinsonian syndromes such as progressive supranuclear palsy (PSP) and multiple system atrophy (MSA) may mimic PD. Thus, biomarkers for early detection of PD are urgently needed to improve the accuracy of early diagnosis to facilitate evaluation of neuroprotective or disease-modifying therapies.

Blood-based biomarkers have great potential because blood captures biological and chemical contents released by cells as waste or as signals to adjacent cells. Sampling from blood is simple, minimally invasive and more feasible compared with, e.g., cerebrospinal fluid (CSF) or tissue biopsies, especially for neurodegenerative disease patients. Global proteomic studies, using blood serum or plasma as a source of biomarker discoveries, have been reported in Alzheimer's disease and, most recently, in PD with cognitive impairment. The development of blood-based biomarkers for PD has great potential but is still in its infancy.

MicroRNAs (miRNAs) are small (18 to 24 nucleotides), highly conserved, genome-encoded single-stranded RNA molecules that bind to target sequences on messenger RNAs (mRNAs), and can act as post-transcriptional regulators of gene expression. MiRNAs are derived from primary miRNA transcripts through processing by the Drosha ribonuclease and the Dicer enzyme. Binding between miRNA and mRNA can repress translation and/or effect decreased mRNA stability in vivo, often by triggering the destruction of the mRNA by endogenous ribonucleases. Regulation of gene expression by miRNA has been found to be important in the normal development and function of most eukaryotic organisms. It has been reported in animal models and cultured neurons that genetically inactivated Dicer can contribute to neurodegeneration. Most recently, a miRNA that regulates the learning processes and may play a central role in Alzheimer's disease has been identified. MiRNA may be differentially expressed in different tissues and different disease states. MiRNAs are known to be abundant, tissue specific, stable in plasma, and have proven feasible to develop as plasma-based biomarkers for diseases such as Huntington's disease, myelodysplastic syndrome, myocardial infarction, and various cancers.

MiRNA may also be expressed or detectable from intracellular environments within the body. Preliminary studies have shown that it is feasible to detect brain-specific miRNAs along with plasma-specific circulating miRNAs in blood plasma. In one preliminary study, 24 brain-specific miRNAs that were originally reported by Kim et. al. [(2007) A MicroRNA feedback circuit in midbrain dopamine neurons. Science 317:1220-1224] along with 54 plasma-specific miRNAs cited by Mitchell et. al. [(2008) Circulating microRNAs as stable blood-based markers for cancer detection. Proc. Natl. Acad. Sci (USA) 105:10513-10518] were detected in healthy control blood plasma. In the same preliminary study, 35 miRNAs expressed in a commercially available human brain tissue reference that could also be detected in healthy control plasma.

### SUMMARY OF THE INVENTION

Global miRNA expression profiles from RNA containing circulating miRNAs in plasma of PD patients and healthy controls were acquired using microarray technology. Comprehensive genomics and bioinformatics analyses were integrated and identified a panel of PD-predictive miRNA biomarkers from plasma samples. Biomarkers capable of discriminating PD patients from healthy controls were validated with real-time quantitative PCR (qRT-PCR). A combination of TSP1 classifier (miR-1826/miR-450b-3p), miR-626, and miR-505 achieved the highest predictive power of 91% sensitivity, 100% specificity, 100% positive predicted value, and 88% negative predicted value. Integrating high-throughput genomic technologies and innovative bioinformatic approaches can identify circulating miRNAs as PD-predictive biomarkers.

In one embodiment, the inventive method of determining whether a subject has Parkinson's disease, is at increased risk of developing Parkinson's disease, or has Parkinson's disease that is progressing or is in remission, may include obtaining a biological sample from the subject; detecting the level of one or more miRNA in the biological sample, wherein the one or more miRNA are selected from the group consisting of miR-505, miR-626, miR-222, miR-1826, miR-450b-3p, miR-1307, miR-647, miR-548b-3p, miR-192*, miR-506, miR-572, miR-671-5p, miR-9*, miR-1225-5p, miR-632, miR-99a*, miR-891b, miR-579, miR-708*, miR-1253, miR-200a, miR-455-3p, miR-192*, miR-485-5p, miR-488, and miR-518c; comparing a level of the one or more miRNA in the biological sample to a statistically validated threshold for each miRNA, which statistically validated threshold for each miRNA is based on the level of the miRNA in comparable control biological samples; and determining that the subject has Parkinson's disease or is at increased risk of developing Parkinson's disease, or that the Parkinson's disease has progressed or is in remission in the subject, when the one or more miRNA are at a different level in the biological sample as compared to the statistically validated threshold for each miRNA.

In some aspects of the method, the detection step may include amplifying any of the one or more miRNA present in the sample, using at least one oligonucleotide primer, to create an amplification product unique to each miRNA, wherein the at least one oligonucleotide primer comprises a region complementary to the miRNA. In some aspects, the oligonucleotide primer comprises cDNA. In some aspects, the detection step may include using an oligonucleotide having a sequence complementary to the miRNA sequence. In some aspects, the oligonucleotide may comprise cDNA In aspects of the method, the oligonucleotide may be a primer.

In some aspects, the amplification product may be a cDNA. In some aspects, the amplification product may be a cDNA having a sequence complementary to any miRNA present in the sample. In some aspects, the amplification step may further include adding RNA bases to one or more ends of the miRNA oligonucleotide.

In some aspects, the detection step further comprises using a probe to detect the presence of any of the miRNA in the biological sample. In some aspects, the probe may be tagged with a detection signal. In some aspects, the probe comprises an oligonucleotide having a sequence that is complementary or identical to all or a region of the miRNA sequence. In some aspects, the probe may be cDNA.

In some aspects of the present invention, the biological sample from the subject may be selected from brain tissue, cerebrospinal fluid, blood plasma, or a combination thereof. In some aspects, the biological sample from the subject may be blood plasma.

In some aspects, the comparable control biological samples are from healthy subjects, and/or from subjects having Parkinson's disease.

In some aspects, it may be determined from the determining step that the subject has Parkinson's disease or is at increased risk of developing Parkinson's disease, or that the Parkinson's disease has progressed or is in remission in the subject initiates a treatment for Parkinson's disease. In some aspects, the method may include treating the subject with a new or modified treatment for Parkinson's disease.

In some aspects of the present invention, the one or more miRNA may be selected from miR-505, miR-626, miR-1307, miR-647, miR-548b-3p, miR-192*, miR-506, miR-1826, miR-572, miR-671-5p, miR-222, miR-9*, and miR-1225-5p; from one or more of the biomarker pairs: miR-1826/miR-450b-3p, miR-1307/miR-632, miR-647/miR-99a*, miR-1225-5p/miR-891b, miR-579/miR-708*, miR-506/miR-1253, miR-200a/miR-455-3p, miR-192*/miR-485-5p, and miR-488/miR-518c*; two or more miRNA selected from one or more of the biomarker pairs miR-1826/miR-450b-3p, miR-506/miR-1253, miR-200a/miR-455-3p, miR-192*/miR-485-5p, and miR-488/miR-518c*; one or more miRNA selected from one or more of miR-505, miR-626, and miR-222; one or more miRNA are selected from: one or more of the biomarker pairs miR-1826/miR-450b-3p, miR-506/miR-1253, miR-200a/miR-455-3p, miR-192*/miR-485-5p, and miR-488/miR-518c*, and/or single miRNA miR-505, miR-626, and miR-222. In some aspects, the miRNA may be miR-505. In some aspects, the miRNA may be the biomarker pair miR-1826/miR-450b-3p. In other aspects, the miRNA may be miR-50, miR-1826, miR-450b-3p, and miR-626.

In another embodiment, the inventive method of treating Parkinson's disease may include obtaining a biological sample from the subject; detecting the level of one or more miRNA in the biological sample, wherein the miRNA is selected from the group consisting of miR-505, miR-626, miR-222, miR-1826, miR-450b-3p, miR-1307, miR-647, miR-548b-3p, miR-192*, miR-506, miR-572, miR-671-5p, miR-9*, miR-1225-5p, miR-632, miR-99a*, miR-891b, miR-579, miR-708*, miR-1253, miR-200a, miR-455-3p, miR-192*, miR-485-5p, miR-488, and miR-518c; comparing a level of the miRNA in the biological sample to a statistically validated threshold for the miRNA, which statistically validated threshold for the miRNA is based on the level of the miRNA in comparable control biological samples; determining that the subject has Parkinson's disease or is at increased risk of developing Parkinson's disease, or that the Parkinson's disease has progressed or is in remission in the subject, when the miRNA is at a different level in the biological sample as compared to the statistically validated threshold for the miRNA, wherein it is determined from the determining step that the has Parkinson's disease or is at increased risk of developing Parkinson's disease, or that the Parkinson's disease has progressed or is in remission in the subject initiates a treatment for Parkinson's disease; and treating the subject with a new or modified treatment for Parkinson's disease.

Another embodiment of the present invention includes a kit for determining whether a subject has Parkinson's disease, is at increased risk of developing Parkinson's disease, or has Parkinson's disease that is progressing or is in remission, including one or more oligonucleotide primers capable of hybridizing to one or more miRNA, wherein the one or more oligonucleotide primer is complementary to one or more miRNA selected from the group consisting of miR-505, miR-626, miR-222, miR-1826, miR-450b-3p, miR-1307, miR-647, miR-548b-3p, miR-192*, miR-506, miR-572, miR-671-5p, miR-9*, miR-1225-5p, miR-632, miR-99a*, miR-891b, miR-579, miR-708*, miR-1253, miR-200a, miR-455-3p, miR-192*, miR-485-5p, miR-488, and miR-518c.

In some embodiments of the kit, the miRNAs are selected from the group consisting of miR-505, miR-1307, miR-647, miR-548b-3p, miR-192*, miR-506, miR-626, miR-1826, miR-572, miR-671-5p, miR-222, miR-9*, and miR-1225-5p; from the group consisting of the miRNA biomarker pairs miR-1826/miR-450b-3p, miR-1307/miR-632, miR-647/miR-99a*, miR-1225-5p/miR-891b, miR-579/miR-708*, miR-506/miR-1253, miR-200a/miR-455-3p, miR-192*/miR-485-5p, and miR-488/miR-518c*; from the group consisting of the miRNA pairs miR-1826/miR-450b-3p, miR-506/miR-1253, miR-200a/miR-455-3p, miR-192*/miR-485-5p, and miR-488/miR-518c*; from the group consisting of miR-505, miR-626, and miR-222; from the group consisting of biomarker pairs miR-1826/miR-450b-3p, miR-506/miR-1253, miR-200a/miR-455-3p, miR-192*/miR-485-5p, and miR-488/miR-518c*, and miR-505, miR-626, miR-222. In some embodiments of the kit, the miRNA is miR-505. In some embodiments of the kit, the miRNA are the biomarker pair miR-1826/miR-450b-3p. In other embodiments, the miRNA are miR505, miR-1826, miR-450b-3p, and miR-626.

### BRIEF DESCRIPTION OF THE DRAWINGS

FIG. 1 depicts the design of the experiment discussed in Examples 1-8.

FIGS. 2A and 2B depict (A) the number of mutually inclusive brain- and plasma-specific miRNAs between published data and a preliminary study of blood plasma miRNA; (B) the number of mutually inclusive brain-specific miRNAs and blood plasma miRNAs from a preliminary study.

FIG. 3 depicts a heat map of microarray miRNA data showing the 9 highest k-TSP PD-predictive miRNAs.

FIG. 4 depicts a heat map of microarray miRNA data showing the 13 most differentially expressed miRNAs as found by SAM analysis.

FIGS. 5A-5C depict a scatterplot of CT values, threshold, and p values of (A) miR-626; (B) miR-505; (C) miR-222.

FIG. 6 depicts a scatterplot of CT and threshold values of miR-505 in the validation subset population. MiR-505 showed lower specificity and sensitivity when samples from individual non-PD control subjects (with MSA and/or PSP) were included in the validation set.

### DETAILED DESCRIPTION OF THE INVENTION

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All references, patents, patent publications, articles, and databases, referred to in this application are incorporated herein by reference in their entirety, as if each were specifically and individually incorporated herein by reference. Such patents, patent publications, articles, and databases are incorporated for the purpose of describing and disclosing the subject components of the invention that are described in those patents, patent publications, articles, and databases, which components might be used in connection with the presently described invention. The information provided below is not admitted to be prior art to the present invention, but is provided solely to assist the understanding of the reader.

The details of one or more embodiments of the invention are set forth in the accompanying drawings and the description below. Other features, embodiments, and advantages of the invention will be apparent from the description and drawings, and from the claims. The preferred embodiments of the present invention may be understood more readily by reference to the following detailed description of the specific embodiments and the Examples included hereafter.

For clarity of disclosure, and not by way of limitation, the detailed description of the invention is divided into the subsections that follow.

### 1. DEFINITIONS

Unless defined otherwise, all technical and scientific terms used herein have the meaning commonly understood by one of ordinary skill in the art to which this invention belongs. Generally, the nomenclature used herein and the laboratory procedures in cell culture, molecular genetics, organic chemistry and nucleic acid chemistry described below are those well-known and commonly employed in the art. Although any methods, devices and materials similar or equivalent to those described herein can be used in the practice or testing of the invention, the preferred methods, devices and materials are now described.

In this specification and the appended claims, the singular forms “a,” “an” and “the” include plural reference unless the context clearly dictates otherwise.

As used in the present application, “biological sample” means any fluid or other material derived from the body of a normal or diseased subject, such as tissue (e.g., brain tissue spinal tissue, liver tissue, etc.), blood, serum, plasma, lymph, urine, saliva, tears, cerebrospinal fluid, milk, amniotic fluid, bile, ascites fluid, pus, and the like. Also included within the meaning of the term “biological sample” is an organ or tissue extract and culture fluid in which any cells or tissue preparation from a subject has been incubated. Methods of obtaining biological samples are well known in the art. Extraction of RNA, e.g., miRNA, from a biological sample such as blood plasma, cerebrospinal fluid, or brain tissue may be performed using well-known methods in the art.

A “diagnosis” of PD may include the early detection of the disease or a confirmation of the diagnosis of the disease from other signs and/or symptoms (e.g., based in whole or in part on expression or expression pattern of one or more miRNA biomarkers). A “diagnosis” of PD may include an assessment of the degree of disease severity (e.g., “low” to “high”; “low- to “high-dependency”), current state of disease progression (e.g., “early”, “middle,” or “late” stages of PD), or include a comparative assessment to an earlier diagnosis (e.g., the PDs symptoms are advancing, stable, or in remission). A diagnosis may include a “prognosis,” that is, a future prediction of the progression of PD, based on the observed disease state (e.g., based in whole or in part on expression or expression pattern of one or more miRNA biomarkers). A diagnosis or prognosis may be based on one or more samplings of miRNA from a biological sample obtained from a subject, and may involve a prediction of disease response to a particular treatment or combination of treatments for PD. An “increased risk” of developing PD may be diagnosed by the presence of expression patterns characteristic of one or more miRNA biomarkers for early or late stage PD in otherwise asymptomatic subjects.

As used herein, “nucleic acids” encompass nucleotides of RNA and DNA, including cDNA (DNA transcribed from RNA template strands), genomic DNA, synthetic (e.g., chemically synthesized) DNA and chimeras of RNA and DNA. The nucleic acid may be double-stranded or single-stranded. Where single-stranded, the nucleic acid may be a sense strand or an antisense strand. The nucleic acid may be synthesized using nucleotide analogs or derivatives (e.g., inosine or phosphorothioate nucleotides). Such oligonucleotides can be used, for example, to prepare nucleic acids that have altered base-pairing abilities or increased resistance to nucleases.

As used herein, the term “oligonucleotide,” refers to a short length of single-stranded polynucleotide chain. Oligonucleotides are typically less than 200 residues long (e.g., between 8 and 100), however, as used herein, the term is also intended to encompass longer polynucleotide chains (e.g., as large as 5000 residues). Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes. Oligonucleotides may be natural e.g., comprising unmodified DNA and/or RNA or synthetic, e.g., modified backbones, modified bases, etc. Examples of oligonucleotide modification are discussed more fully below.

As used herein, the terms “complementary” or “complementarity” are used in reference to oligonucleotides related by the base-pairing rules for DNA-DNA, RNA-DNA and RNA-RNA pairing. For example, for the sequence “A-G-T,” is complementary to the sequence “T-C-A.” Complementarity may be “partial,” in which only some of a pair of nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Under “low stringency” conditions, strands with a lower degree of complementarity will hybridize with each other. Under “high stringency conditions,” only strands with a higher degree of complementarity will remain hybridized with each other.

As used herein, the term “completely complementary,” refers to an oligonucleotide where all of the nucleotides are complementary to a target sequence (e.g., a miRNA). A completely complementary oligonucleotide may be shorter than the target sequence, thus, only hybridizing to a portion of the target.

As used herein, the term “partially complementary” refers to an oligonucleotide where at least one nucleotide is not complementary to (i.e., one or more “mismatches” with) the target sequence. Preferred partially complementary oligonucleotides are those that can still hybridize to a target sequence under physiological conditions. A particular partially complementary oligonucleotide may have a ‘random’ pattern of one or more mismatches with the target sequence throughout the oligonucleotide (although the pattern of mismatches is preferentially constrained by retention of the ability to still hybridize to the target sequence under physiological conditions). A particular partially complementary oligonucleotide may have regions where the oligonucleotide sequence is highly, or even completely complementary to a target sequence, and regions where the oligonucleotide is not complementary, or is less complementary to the target sequence.

For example, partially complimentary oligonucleotides may have one or more regions that hybridize to a target sequence, and one or more regions that do not hybridize to the target sequence. Thus, a partially complementary sequence (such as a PCR or reverse transcriptase (RT) primer) may hybridize to a portion (i.e., the middle, the 5′, or 3′ end) of a particular target sequence, and not hybridize with the rest of the target sequence. Oligonucleotides with mismatches at the ends may still hybridize to the target sequence. Partially complementary sequences may be capable of binding to a sequence having less than 60%, 70%, 80%, 90%, 95%, to less than 100% identity to the target sequence. For purposes of defining or categorizing partially complementary sequences, a partially complementary sequence or region of a sequence becomes more complementary or becomes “highly complementary” as it approaches 100% complementarity to a target sequence. Thus, a highly complementary sequence may have 60%, 70%, 80%, 90%, 95%, to 99% identity to all or a portion of a target sequence. The exact percentage identity of the highly complementary sequence may depend on the length of the highly complementary sequence and the desired stringency and specificity of hybridization. Partially complementary sequences may hybridize to one or more target sequences.

As noted above, partially complementary sequences may be completely complementary or highly complementary to a portion of the target sequence, such that they are completely or highly complementary to, e.g., 5%, 10%, 20%, 30%, 40%, 50% 60%, 70%, 80%, 90%, 95%, 99% of the target sequence. Similarly, 5%, 10%, 20%, 30%, 40%, 50% 60%, 70%, 80%, 90%, 95%, 99% of the partially complementary sequence may be completely complementary or highly complementary to all or a portion of the target sequence.

In the context of reverse transcriptase or PCR primers, detection probes, and the like, 5, 6, 7, 8, 9, 10, 11, 12, 13, 14, 15, etc. contiguous base pairs may be completely complementary or highly complementary to a target oligonucleotide sequence. In some cases, an entire probe may be completely complementary to a portion of (i.e., is shorter than) a target sequence.

“Identical” oligonucleotides may be completely or substantially share the same base sequence as a target sequence. “Identical” oligonucleotides may comprise a region or regions that are completely or substantially identical to all or a portion of a target sequence, and regions that are not identical. Often, ‘identical’ oligonucleotides are synthesized (using, e.g., RNA or DNA polymerase enzymes) from sequences that are completely or partially complementary to a target sequence. A DNA oligonucleotide may be completely or substantially identical to a RNA target oligonucleotide with the exception that the uracil (U) bases of the RNA target oligonucleotide are thymidine (T) in the DNA oligonucleotide. Similarly, an RNA oligonucleotide may be completely or substantially identical to a DNA target oligonucleotide with the exception that the thymidine (T) bases of the DNA target oligonucleotide are uracil (U) in the RNA oligonucleotide.

The term “homology” refers to a degree of complementarity. There may be partial homology or complete homology (i.e., identity). A partially complementary oligonucleotide that may at least partially inhibit a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized,” and may form stem and loop structures or the like under certain conditions.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest is capable of hybridizing to its exact complement, sequences with single base mismatches, closely related sequences (e.g., sequences with 90, 91, 92, 93, 94, 95, 96, 97, 98, 99% or greater homology), and sequences having only partial homology (e.g., sequences with 50-90% homology). Under “medium stringency conditions,” a nucleic acid sequence of interest capable of hybridizing only to its exact complement, sequences with single base mismatches, and closely relation sequences (e.g., 91, 92, 93, 94, 95, 96, 97, 98, 99% or greater homology). Under “high stringency conditions,” a nucleic acid sequence of interest capable of hybridizing only to its exact complement, and (depending on conditions such a temperature) sequences with single base mismatches, in other words, under conditions of high stringency, the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H2O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H2O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l aH PO4H2O and 1.85 g/l EDTA, pH adjusted to 7.4 with NaOH), 0.1% SDS, SX Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The present invention is not limited to the hybridization of primers or probes of about 500 nucleotides in length. The present invention contemplates the use of primers or probes between approximately 4-5 nucleotides up to several thousand (e.g., at least 5000) nucleotides in length. One skilled in the relevant understands that stringency conditions may be altered for primer or probes of other sizes (See e.g., Anderson and Young, Quantitative Filter Hybridization, in Nucleic Acid Hybridization [1985] and Sambrook et al, Molecular Cloning: A Laboratory Manual, Cold Spring Harbor Press, NY [1989]).

In is well known in the art that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “physiological conditions” refers to specific stringency conditions that approximate or are conditions inside an animal (e.g., a human). Exemplary physiological conditions for use in vitro include, but are not limited to, 37° C., 95% air, 5% CO2, commercial medium for culture of mammalian cells (e.g., DMEM media available from Gibco, MD), 5-10% serum (e.g., calf serum or horse serum), additional buffers, and optionally hormone (e.g., insulin and epidermal growth factor).

As used herein the term “region” when in reference to an oligonucleotide (as in “a region of a given oligonucleotide”) refers to a contiguous portion of the entire oligonucleotide sequence. Regions may range in size from 3-4 bases to the entire sequence minus one base.

As used herein, a “primer” means an oligonucleotide sequence that is capable of initiating or facilitating transcription or translation of a template oligonucleotide by binding or hybridizing to a template or target oligonucleotide. In some instances, a primer may contain one or more sequences that are complementary to the template or target oligonucleotide.

As used herein, a “probe” is used to detect the presence of an oligonucleotide in a sample, often by selectively binding or hybridizing to all or portion of the oligonucleotide sequence. In some aspects, probes may be oligonucleotides, antibodies, and the like. Probes may be “tagged” with a detection label to aid in observation or detection of the oligonucleotide. “Tagging” may include covalent, ionic, hydrogen or other chemical bonding to a fluorophore, radiolabelling a portion or one or more atoms of the probe, and the like. Examples of probes and tagging techniques are provided more fully below.

As used herein, the term “microRNA” (miRNA) includes primary miRNA transcripts (pri-miRNA) or other mRNA transcripts that code for mature miRNA (e.g., miRNA processed from introns excised from mRNA transcripts), precursor miRNAs (pre-miRNA), mature single stranded miRNAs, and variants thereof, which may be naturally occurring. The term “miRNA” includes human and miRNA from other eukaryotic organisms. In some instances, the term “miRNA” also includes primary miRNA transcripts and duplex miRNAs. Unless otherwise noted, when used herein, the name of a specific miRNA refers to the mature miRNA of a precursor miRNA. Some single primary miRNA transcripts may contain more than one precursor/mature miRNA. Some mature miRNA may be derived from more than one precursor miRNA.

In most eukaryotes, primary miRNA is transcribed from DNA, and is processed by cellular machinery to form precursor miRNA, which is further processed to form one or more miRNA. In some eukaryotes and for some miRNA, precursor miRNA is transcribed directly from DNA, and is further processed to form miRNA (e.g., processed from introns excised from mRNA transcripts). Unless otherwise noted, the name of a specific miRNA refers to a mature miRNA sequence. Under current nomenclature rules, human miRNA are preceded with the prefix “hsa-” (i.e., an abbreviation for Homo sapiens). Throughout the specification and figures the hsa- prefix may be dropped for purposes of abbreviation, thus, for example, ‘hsa-miR-1307’ and ‘miR-1307’ would represent the same RNA sequence. Sequences from other species, where present, are labeled as such.

As used herein, “cDNA” refers to DNA oligonucleotide sequences that are completely or partially complementary; or completely or partially/substantially identical to RNA sequences (e.g., a miRNA sequence). As is well known the art, cDNA oligonucleotides may be generated directly from RNA oligonucleotides using the RNA sequence as a template, often by the use of a reverse transcriptase enzyme. cDNA may also be designed and directly synthesized using any sequencing method known in the art. The term “cDNA” includes sequences both (completely or partially) complementary to an RNA sequence and (completely or partially/substantially) identical to an RNA sequence (given the substitution of thymine (T) base for the uracil (U) present in the RNA sequence). cDNA sequences identical to the RNA sequence are often produced using a cDNA oligonucleotide with a complementary sequence as a template, for instance, using PCR, or any other synthetic method known in the art.

The term “amplification product” refers to an oligonucleotide sequence that whose copy number (i.e., concentration) has been increased using a amplification reaction, such as, for example, the polymerase chain reaction (PCR).

The term “subject” or “patient” as used herein refers to a mammal, preferably a human, in need of diagnosis and/or treatment for a condition, disorder or disease.

### 2. PARKINSON'S DISEASE

Most symptoms of PD are caused by an absence or suboptimal levels of dopamine in the brain, in some instance caused by a degeneration of dopaminergic neurons over time. Primary symptoms include motor dysfunction, and may also include non-motor manifestations as well.

There are currently no direct cures for Parkinson's disease. Drugs and surgical therapies are most often used to lessen the symptoms of PD such as muscle rigidity and tremor and improve overall movement and coordination. Dopaminergic drugs may be used (e.g., dopamine-replenishing or dopamine mimicking) to temporarily boost the levels of dopamine or dopamine response in the brain or other neural tissue, Such drugs include levodopa treatments, levodopa, or levodopa combination treatments, which may include, administration with carbodopa, a dopamine enhancer, and/or the COMT-inhibitor entacapone; dopamine agonists, including ropinirole, pramipexole, rotigotine, apomorphine, pergolide and bromocriptine; MAO-B inhibitors, which are used alone or with levodopa, including selegiline, rasagiline, zydis selegiline HCl salt; COMT-inhibitors used to boost the effects of levodopa include entacapone, tolcapone; amantadine, often used to combat tremor and side effects of levodopa administration; anti-cholinergenics such as trihexyphenidyl, and benztropine.

Other drugs currently under study include antiglutamatergics such as memantine, safinomide; neurturin therapies; anti-apoptotics, such as omigapil and CEP-1347; promitochondrials, such as CoQ (Coenzyme Q10) and creatine; calcium channel blockers, including isradipine, and growth factors such as GDNF; as well as drugs or vaccines targeting alpha-synuclein.

Surgical therapies may include deep brain stimulation (DBS), involving implantation of a battery-powered electrode in the brain; operations directly on neural tissue including, thalamotomy; pallidotomy; subthalmatomy; dopamergic cell transplant.

Diet and exercise regimen may alleviate PD symptoms. Complementary or alternative therapies such as administration of herbal supplements alone or in combination with one of the above drug or surgical therapies may be used in some instances. Supplements may include antioxidants such as vitamins C and E, calcium, ginger root, green tea and green tea extracts, St. John's Wort, Ginkgo biloba, milk thistle, vitamin B12, and folic acid. Other atypical Parkinsonian syndromes or “Parkinsons-plus” syndromes are not usually responsive to dopaminergic agents such as levodopa, but otherwise mimic or have symptoms similar to PD. Atypical Parkinsonian syndrome such as progressive supranuclear palsy (PSP), multiple system atrophy (MSA), corticobasal degeneration (CBGD), Pick's disease, olivopontocerebellar atrophy (OPCA) may be diagnosed or distinguished from PD using biomarkers of the present invention.

### 3. ACQUISITION OF BIOMARKERS FOR PD

The inventors have been able to differentiate PD patients from healthy individuals based on circulating miRNAs in the plasma. Not to be limited by theory, it is thought that dopaminergic neurons of Parkinson's disease (PD) patients release biological and chemical contents, including miRNA, into the blood. Thus, in some aspects of the invention, methods of diagnosing and prognosing PD may include detecting levels (amounts) of one or more miRNA biomarkers in a biological sample from a subject or a patient, comparing those levels with a statistically validated threshold for each miRNA, and determining whether the subject or patient has PD, is prone to having PD, or whether PD is in progression or remission in the subject or patient. In some aspects, detecting the levels of one or more miRNA involves amplification and detection of an oligonucleotide (e.g., cDNA) complementary to the miRNA biomarker.

In general, suitable biomarkers may be any miRNA that shows a differential level of expression or concentration in biological samples of healthy and diseased subjects, or in biological samples of PD patients at different stages in the disease. Preferred miRNA biomarkers have a more pronounced difference between individual healthy and diseased subjects and/or a low variability between populations of individual healthy and diseased subjects, which reduces the likelihood of both false positives and negatives, and improves the sensitivity and specificity of the diagnosis and/or prognosis.

Further, preferred miRNA biomarkers have good reproducibility, and are relatively easy to extract and amplify using a chosen detection protocol. In some embodiments of the invention, more than one miRNA biomarker may be used in a diagnostic test to give improved sensitivity, specificity, and positive and negative predictive value.

The terms “differentially expressed,” “reduced levels” or “elevated levels” refer to the amount of expression or concentration of a miRNA in a biological sample from a patient compared to the amount of the miRNA in biological sample from individual(s) that do not have PD, have PD (or a particular severity or stage of PD), or have other reference diseases, such as Alzheimer's Disease, or PD-plus diseases such as PSP and MSA. For example, a miRNA that has reduced levels in a biological sample from PD patients is present at lower concentration in biological sample from PD patients than in serum from a subject who does not have PD. For certain miRNAs, elevated levels in a biological sample indicates the presence of or a risk for PD; at the same time, other miRNAs may be present in reduced levels in patients or subjects with PD. In either of these example situations, miRNAs are “differentially expressed” in PD subjects and healthy controls.

Individual, differentially expressed miRNA biomarkers may be found by expression screening technologies such as miRNA microarrays that may contain large numbers or a “library” of miRNA sequences. Typically, miRNAs obtained from biological samples from a population of healthy subjects and a population of individuals with a disease of interest may be run on a microarray, and the differential expression of some or all of the members of the miRNA library in each sample is assessed. Other screening/high-throughput technologies that may be used to quantify and differentiate miRNA expression include massively parallel Next Generation Sequencing (NGS) technologies, or qRT-PCR. High-throughput NGS technologies, for instance, can be used to assay entire sets of RNA transcripts within biological samples, and can be used to compare RNA transcription profiles between biological samples. Microarrays and other screening technologies such as NGS may measure the presence/absence of a miRNA in a sample; sequence changes in a particular miRNA; the number of miRNA expressed below and/or above a certain concentration threshold in a sample; or an assessment of the relative or absolute amount of a particular miRNA in a sample.

Typically, microarrays and other expression screening technologies such as NGS generate large amounts of data and finding miRNA expression differences between healthy and diseased populations usually requires statistical analysis. Single mRNA biomarker candidates may be found using Significance Analysis of Microarrays (SAM) protocol, or other protocols known in the art.

Additional biomarker candidates may be found by using pairwise scoring techniques, generating unique pairs of miRNA sequences that are differentially expressed in healthy and diseased populations. One such technique, the K-TSP (k-Top Scoring Pair) is described briefly below.

1. To train the PD-Predictive classifier using k-TSP algorithm: For every miRNA pair (i, j), find the probability of (i>j) in diseased individuals (P1). Next, find the probability of (i>j) in healthy individuals (P2). Compute the score, Sᵢⱼ, for (i, j) pair by taking the absolute difference between P1 and P2. Repeat steps 1-3 for all possible combination of miRNA pairs. Next, rank Sᵢⱼ from the largest to the smallest values; the top pair with the highest Sᵢⱼ will be the 1-top scoring pair. Next, iteratively go down the Sᵢⱼ ranked list to pick up disjoint miRNA pairs. The number of miRNA pairs is determined by an internal leave-one-out cross-validation.

2. To test the PD-Predictive classifier using k-TSP algorithm: To test the k-TSP classifier, simply determine the relative expression of (i, j) miRNA pair in the test sample. Every miRNA pair (i, j) is an independent predictor that makes a prediction on the new test sample (diseased or healthy). If more than one miRNA pairs, the final decision is based on majority voting.

Sequences of miRNA PD biomarkers of the present invention are given below in Table 1.

TABLE 1
Sequences of PD-predictive miRNAs

| Name | Sequence | Identifier |
|---|---|---|
| hsa-miR-1307: | ACUCGGCGUGG | [SEQ ID NO: 1] |
| | CGUCGGUCGUG | |
| hsa-miR-632: | GUGUCUGCUUC | [SEQ ID NO: 2] |
| | CUGUGGGA | |
| hsa-miR-647: | GUGGCUGCACU | [SEQ ID NO: 3] |
| | CACUUCCUUC | |
| hsa-miR-99a*: | CAAGCUCGCUU | [SEQ ID NO: 4] |
| | CUAUGGGUCUG | |
| hsa-miR-1225-5p: | GUGGGUACGGC | [SEQ ID NO: 5] |
| | CCAGUGGGGGG | |
| hsa-miR-891b: | UGCAACUUACC | [SEQ ID NO: 6] |
| | UGAGUCAUUGA | |
| hsa-miR-1826: | AUUGAUCAUCG | [SEQ ID NO: 7] |
| | ACACUUCGAAC | |
| | GCAAU | |
| hsa-miR-450b-3p: | UUGGGAUCAUU | [SEQ ID NO: 8] |
| | UUGCAUCCAUA | |
| hsa-miR-579: | UUCAUUUGGUA | [SEQ ID NO: 9] |
| | UAAACCGCGAUU | |
| hsa-miR-708*: | CAACUAGACUG | [SEQ ID NO: 10] |
| | UGAGCUUCUAG | |
| hsa-miR-506: | UAAGGCACCCU | [SEQ ID NO: 11] |
| | UCUGAGUAGA | |
| hsa-miR-1253: | AGAGAAGAAGA | [SEQ ID NO: 12] |
| | UCAGCCUGCA | |
| hsa-miR-200a: | UAACACUGUCU | [SEQ ID NO: 13] |
| | GGUAACGAUGU | |
| hsa-miR-455-3p: | GCAGUCCAUGG | [SEQ ID NO: 14] |
| | GCAUAUACAC | |
| hsa-miR-192*: | CUGCCAAUUCC | [SEQ ID NO: 15] |
| | AUAGGUCACAG | |
| hsa-miR-485-5p: | AGAGGCUGGCC | [SEQ ID NO: 16] |
| | GUGAUGAAUUC | |
| hsa-miR-488: | UUGAAAGGCUA | [SEQ ID NO: 17] |
| | UUUCUUGGUC | |
| hsa-miR-518c*: | UCUCUGGAGGG | [SEQ ID NO: 18] |
| | AAGCACUUUCUG | |
| hsa-miR-548b-3p: | CAAGAACCUCA | [SEQ ID NO: 19] |
| | GUUGCUUUUGU | |
| hsa-miR-505 | CGUCAACACUU | [SEQ ID NO: 20] |
| | GCUGGUUUCCU | |
| hsa-miR-506: | UAAGGCACCCU | [SEQ ID NO: 21] |
| | UCUGAGUAGA | |
| hsa-miR-626: | AGCUGUCUGAA | [SEQ ID NO: 22] |
| | AAUGUCUU | |
| hsa-miR-572: | GUCCGCUCGGC | [SEQ ID NO: 23] |
| | GGUGGCCCA | |
| hsa-miR-671-5p: | AGGAAGCCCUG | [SEQ ID NO: 24] |
| | GAGGGGCUGGAG | |
| hsa-miR-222: | AGCUACAUCUG | [SEQ ID NO: 25] |
| | GCUACUGGGU | |
| hsa-miR-9*: | AUAAAGCUAGA | [SEQ ID NO: 26] |
| | UAACCGAAAGU | |

In some aspects of the invention, one or more miRNA may be used as a biomarker for PD. Single miRNA suitable for use in this invention include one or more of miR-1307, miR-647, miR-548b-3p, miR-192*, miR-505, miR-506, miR-626, miR-1826, miR-572, miR-671-5p, miR-222, miR-9*, and miR-1225-5p, which may be used alone or with other miRNA biomarkers or biomarker pairs.

In other aspects of the invention, one or more miRNA pairs may be used. MiRNA pairs suitable for use in this invention include miR-1307/miR-632, miR-647/miR-99a*, miR-1225-5p/miR-891b, miR-1826/miR-4506-3p, miR-579/miR-708*, miR-506/miR-1253, miR-200a/miR-455-3p, miR-192*/miR-485-5p, and miR-488/miR-518c*, which may be used alone or with other miRNA biomarkers or biomarker pairs.

In other aspects of the invention, two or more single miRNA (e.g., two, three, three or more, four, four or more, five, five or more, six, six or more, seven, seven or more, eight, eight or more, nine, nine or more, ten, ten or more, eleven, eleven or more, twelve, twelve or more, thirteen) may be used as biomarkers for PD, selected from miR-1307, miR-647, miR-548b-3p, miR-192*, miR-505, miR-506, miR-626, miR-1826, miR-572, miR-671-5p, miR-222, miR-9*, and miR-1225-5p.

In other aspects of the invention, two or more miRNA pairs (e.g., two, three, three or more, four, four or more, five, five or more, six, six or more, seven, seven or more, eight, eight or more, nine) may be used as biomarkers for PD, selected from miR-1307/miR-632, miR-647/miR-99a*, miR-1225-5p/miR-891b, miR-1826/miR-450b-3p, miR-579/miR-708*, miR-506/miR-1253, miR-200a/miR-455-3p, miR-192*/miR-485-5p, and miR-488/miR-518c*.

In other aspects of the invention, at least one single miRNA (e.g., two, three, three or more, four, four or more, five, five or more, six, six or more, seven, seven or more, eight, eight or more, nine, nine or more, ten, ten or more, eleven, eleven or more, twelve, twelve or more, thirteen) and at least one pair of miRNAs (e.g., two, three, three or more, four, four or more, five, five or more, six, six or more, seven, seven or more, eight, eight or more, nine) may be used as biomarkers for PD, selected from miR-1307, miR-647, miR-548b-3p, miR-192*, miR-505, miR-506, miR-626, miR-1826, miR-572, miR-671-5p, miR-222, miR-9*, and miR-1225-5p; and the pairs miR-1307/miR-632, miR-647/miR-99a*, miR-1225-5p/miR-891b, miR-1826/miR-450b-3p, miR-579/miR-708*, miR-506/miR-1253, miR-200a/miR-455-3p, miR-192*/miR-485-5p, and miR-488/miR-518c*.

In some aspects one or more miRNA biomarkers are selected from the pairs: miR-1826/miR-450b-3pTSP1, miR-506/miR-1253 TSP2, miR-200a/miR-455-3p, miR-192*/miR-485-5p, and miR-488/miR-518c*. In some aspects, aspects one or more miRNA biomarkers are selected from miR-626, miR-222, and miR-505. In some aspects, one or more miRNA biomarkers are selected from the biomarker pairs miR-1826/miR-450b-3p, miR-506/miR-1253, miR-200a/miR-455-3p, miR-192*/miR-485-5p, and miR-488/miR-518c*, and single miRNA miR-626, miR-222, and miR-505.

In yet another aspect of the method, the miRNA biomarker is miR-505. In yet another aspect of the method, the miRNA biomarker is miR-626. In other aspects, the miRNA biomarker pair is miR-1826/miR-450b-3p. In other aspects, the miRNA biomarkers are miR-1826/miR-450b-3p, miR-626, and miR-505.

### 4. DETECTION OF BIOMARKERS IN BIOLOGICAL SAMPLES

Specific miRNA biomarkers may be detected and/or quantified with any detection method or technique known in the art, for example, but not limited to, microarrays, NGS, or qRT-PCR. Methods may detect the presence or absence of a particular miRNA, sequence changes of a particular miRNA, presence of level (i.e., an amount) of a specific miRNA above a detection threshold or a diagnostic threshold correlated with the presence or absence of a disease, or determine the concentration of specific miRNA in a particular sample.

Any method of measuring or quantitating the amount of miRNA in a biological sample can be used. Preferred methods are reliable, sensitive and specific for a particular miRNA used as a biomarker in aspects of the present invention. Methods such as differential display, RNAase protection assays and Northern or Southern blots may be used to quantify miRNA in a biological sample, or indirectly quantify miRNA in a biological sample through amplification and detection of cDNA oligonucleotides (completely or partially) complementary or (completely or partially) identical to the miRNA biomarker. More recently developed techniques such as qRT-PCR offer more sensitive and less labor-intensive quantification of miRNA in samples. MiRNA may or may not be amplified by techniques such as polymerase chain reaction (PCR) prior to measurement, or the quantity of miRNA may be directly or indirectly measured during amplification. Quantitative assessments such as real-time quantitative PCR (qRT-PCR) assay are simple, sensitive, reproducible, and cost-effective; hence they are suitable to use as a diagnostic tool. Example amplification and detection techniques are described below.

A. Amplification

For purposes of detection and subsequent quantification, the concentration of miRNA, or cDNA amplification products, may be amplified as part of the detection and amplification process. In many amplification processes, amplification proceeds in a predictable fashion over time, such that final concentrations of amplification products can be used to determine initial concentration of particular miRNA in a sample, prior to amplification.

Several exemplary methods exist for amplifying miRNA nucleic acid sequences such as mature miRNAs, precursor miRNAs, and primary miRNAs. Suitable nucleic acid polymerization and amplification techniques include reverse transcription (RT), polymerase chain reaction (PCR), real-time PCR (quantitative PCR (q-PCR), nucleic acid sequence-base amplification (NASBA), ligase chain reaction, multiplex ligatable probe amplification, invader technology (Third Wave), rolling circle amplification, in-vitro transcription (IVT), strand displacement amplification, transcription-mediated amplification (TMA), RNA (Eberwine) amplification, and other methods that are known to persons skilled in the art. In certain embodiments, more than one amplification method is used, such as reverse transcription followed by real time quantitative PCR (qRT-PCR) (See, e.g., Chen et al., Nucleic Acids Research, 33(20):e179 (2005); Benes and Castoldi, Methods 50 (2010), pg 244-249).

A typical PCR reaction includes multiple amplification steps, or cycles that selectively amplify target nucleic acid species: a denaturing step in which a target nucleic acid is denatured; an annealing step in which a set of PCR primers (forward and reverse primers) anneal to complementary DNA strands; and an elongation step in which a thermostable DNA polymerase elongates the primers. By repeating these steps multiple times, a DNA fragment is amplified to produce an amplicon, corresponding to the target DNA sequence. Typical PCR reactions include 20 or more cycles of denaturation, annealing, and elongation. In many cases, the annealing and elongation steps can be performed concurrently, in which case the cycle contains only two steps. Since mature miRNAs are single-stranded, a reverse transcription reaction (which produces a complementary cDNA sequence) may be performed prior to PCR reactions. Reverse transcription reactions include the use of, e.g., a RNA-based DNA polymerase (reverse transcriptase) and a primer.

In some embodiments, two or more miRNAs or cDNAs are amplified in a single reaction volume. One aspect includes multiplex q-PCR, such as qRT-PCR, which enables simultaneous amplification and quantification of at least two miRNAs of interest in one reaction volume by using more than one pair of primers and/or more than one probe. The primer pairs comprise at least one amplification primer that uniquely binds each miRNA, and the probes are labeled such that they are distinguishable from one another, thus allowing simultaneous quantification of multiple miRNAs. Multiplex qRT-PCR has research and diagnostic uses, including but not limited to detection of miRNAs for diagnostic, prognostic, and therapeutic applications.

As noted above, the q-PCR reaction may further be combined with the reverse transcription reaction (i.e., RT-PCR) by including both a reverse transcriptase and a DNA-based thermostable DNA polymerase. When two polymerases are used, a “hot start” approach may be used to maximize assay performance (U.S. Pat. Nos. 5,411,876 and 5,985,619). For example, the components for a reverse transcriptase reaction and a PCR reaction may be sequestered using one or more thermoactivation methods or chemical alteration to improve polymerization efficiency (U.S. Pat. Nos. 5,550,044, 5,413,924, and 6,403,341).

For ease of reference, several examples of the types of primers used in reverse transcription and PCR are noted below. The use of primers in qRT-PCR to detect miRNA is discussed in Benes and Castoldi, Methods, 50 (2010) pp. 244-249, and is incorporated by reference herein.

Reverse Transcription.

As noted above, both the reverse transcription reaction and PCR amplification reactions require the use of primers. The primers themselves usually comprise DNA. Reverse transcription primers may be specific to an miRNA sequence, or universal primers may be used. If universal primers are used, a common template sequence must be attached to each miRNA. One way to do so is to build, for example, a polyA tail to each miRNA via the use of polyA polymerase enzymes. Another approach is to ligate a RNA tail sequence to miRNA using a RNA ligase enzyme (see, e.g., Benes and Castoldi; and EP 09002587.5).

Reverse transcriptase primers may be designed to bind with miRNA modified with a common signal, or directly to the miRNA, such that the primer is complementary to a portion or region of the original miRNA sequence, or to the common template sequence. Often, the cDNA primer sequence will include a portion that does not hybridize to the miRNA sequence. As the cDNA primer becomes incorporated into the complementary cDNA strand made by reverse transcriptase, this portion can often comprise a universal PCR template region that is used to bind a PCR primer.

PCR.

PCR and qPCR amplification of the resultant cDNA strand requires two PCR primers to initiate replication of the target cDNA sequence, one hybridizing to the 3′ end of the cDNA generated by reverse transcription (reverse primer), and one hybridizing to the 3′ prime end (forward) of the strand complementary to the cDNA strand (and identical to the miRNA). In certain embodiments, the reverse transcriptase primer may serve as a forward or primer in pCR. In certain embodiments, the lengths of the primers depends on many factors, including, but not limited to, the desired hybridization temperature between the primers, the composition of target nucleic acid sequence, and the complexity of the different target nucleic acid sequences to be amplified, and the amount of correspondence (more than one target sequence is to be amplified in the same reaction) with other target sequences.

In certain embodiments, a primer is about 15 to about 35 nucleotides in length. In other embodiments, a primer is equal to or fewer than 15, 20, 25, 30, or 35 nucleotides in length. In additional embodiments, a primer is at least 35 nucleotides in length.

In a further aspect, a forward primer can comprise at least one sequence that anneals to a miRNA biomarker (or cDNA with the identical sequence) (that is, has a region that is completely or highly complementary to a region of the miRNA biomarker) and alternatively can comprise an additional 5′ non-complementary region. In another aspect, a reverse primer can be designed to anneal to the complementary cDNA of a reverse transcribed miRNA. The reverse primer may be independent of the miRNA biomarker sequence (i.e., where it binds to a universal PCR template region), and multiple miRNA biomarkers may be amplified using the same reverse primer. Alternatively, a reverse primer may be specific for a miRNA biomarker (or cDNA), and be completely or highly (partially) complementary or contain a region that is completely or highly complementary to a region or the entire miRNA biomarker or cDNA.

The region of a forward or reverse PCR or reverse transcriptase primer that is complementary to the target sequence and anneals to a target sequence may be 3, 4, 5, 6, 7, 8, 9, 10, 11, 12, 13, 14, 15 base pairs in length. A region of a forward or reverse PCR or reverse transcriptase primer may be completely or partially complementary to a specific sequence on the template strand.

B. Detection

In certain embodiments, labels, dyes, or labeled probes and/or primers are used to detect amplified or unamplified miRNAs or completely or partially complementary or completely or partially identical cDNAs. The skilled artisan will recognize which detection methods are appropriate based on the sensitivity of the detection method and the abundance of the target. Depending on the sensitivity of the detection method and the abundance of the target, amplification may or may not be required prior to detection. One skilled in the art will recognize the detection methods where miRNA amplification is preferred.

In some aspects, probes and or primers to detect amplified or unamplified miRNAs or completely or partially complementary or completely or partially identical cDNAs may be oligonucleotides having sequences completely or partially complementary or completely or partially identical to the amplified miRNA or cDNA. In one aspect, the probe and/or primer is a cDNA. In another aspect, the probe and or primer is labeled to aid in the detection of the amplified or unamplified miRNAs or completely or partially complementary or completely or partially identical cDNAs.

A probe or primer may include Watson-Crick bases or modified bases. Modified bases include, but are not limited to, the AEGIS bases (from Eragen Biosciences), which have been described, e.g., in U.S. Pat. Nos. 5,432,272, 5,965,364, and 6,001,983. In certain aspects, bases are joined by a natural phosphodiester bond or a different chemical linkage. Different chemical linkages include, but are not limited to, a peptide bond or a Locked Nucleic Acid (LNA) linkage, which is described, e.g., in U.S. Pat. No. 7,060,809.

In a further aspect, oligonucleotide probes or primers present in an amplification reaction are suitable for monitoring the amount of amplification product produced as a function of time. In certain aspects, probes having different single stranded versus double stranded character are used to detect the nucleic acid. Probes include, but are not limited to, the 5′-exonuclease assay (e.g., TaqMan™) probes (see U.S. Pat. No. 5,538,848), stem-loop molecular beacons (see, e.g., U.S. Pat. Nos. 6,103,476 and 5,925,517), stemless or linear beacons (see, e.g., WO 9921881, U.S. Pat. Nos. 6,485,901 and 6,649,349), peptide nucleic acid (PNA) Molecular Beacons (see, e.g., U.S. Pat. Nos. 6,355,421 and 6,593,091), linear PNA beacons (see, e.g. U.S. Pat. No. 6,329,144), non-FRET probes (see, e.g., U.S. Pat. No. 6,150,097), Sunrise™/AmplifluorB™ probes (see, e.g., U.S. Pat. No. 6,548,250), stem-loop and duplex Scorpion™ probes (see, e.g., U.S. Pat. No. 6,589,743), bulge loop probes (see, e.g., U.S. Pat. No. 6,590,091), pseudo knot probes (see, e.g., U.S. Pat. No. 6,548,250), cyclicons (see, e.g., U.S. Pat. No. 6,383,752), MGB Eclipse™ probe (Epoch Biosciences), hairpin probes (see, e.g., U.S. Pat. No. 6,596,490), PNA light-up probes, antiprimer quench probes (Li et al., Clin. Chem. 53:624-633 (2006)), self-assembled nanoparticle probes, and ferrocene-modified probes described, for example, in U.S. Pat. No. 6,485,901.

In certain embodiments, one or more of the primers in an amplification reaction can include a label. In yet further embodiments, different probes or primers comprise detectable labels that are distinguishable from one another. In some embodiments a nucleic acid, such as the probe or primer, may be labeled with two or more distinguishable labels.

In some aspects, a label is attached to one or more probes and has one or more of the following properties: (i) provides a detectable signal; (ii) interacts with a second label to modify the detectable signal provided by the second label, e.g., FRET (Fluorescent Resonance Energy Transfer); (iii) stabilizes hybridization, e.g., duplex formation; and (iv) provides a member of a binding complex or affinity set, e.g., affinity, antibody-antigen, ionic complexes, hapten-ligand (e.g., biotin-avidin). In still other aspects, use of labels can be accomplished using any one of a large number of known techniques employing known labels, linkages, linking groups, reagents, reaction conditions, and analysis and purification methods.

MiRNAs or cDNAs can be detected by direct or indirect methods. In a direct detection method, one or more miRNAs or cDNAs are detected by a detectable label that is linked to a nucleic acid molecule. In such methods, the miRNAs or cDNAs may be labeled prior to binding to the probe. Therefore, binding is detected by screening for the labeled miRNA or cDNA that is bound to the probe. The probe is optionally linked to a bead in the reaction volume.

In certain embodiments, nucleic acids are detected by direct binding with a labeled probe, and the probe is subsequently detected. In one embodiment of the invention, the nucleic acids, such as amplified miRNAs or cDNAs, are detected using FIexMAP Microspheres (Luminex) conjugated with probes to capture the desired nucleic acids.

Some methods may involve detection with polynucleotide probes modified with fluorescent labels or branched DNA (bDNA) detection, for example.

In other embodiments, nucleic acids are detected by indirect detection methods. For example, a biotinylated probe may be combined with a stretavidin-conjugated dye to detect the bound nucleic acid. The streptavidin molecule binds a biotin label on amplified miRNA or cDNA, and the bound miRNA is detected by detecting the dye molecule attached to the streptavidin molecule. In one embodiment, the streptavidin-conjugated dye molecule comprises Phycolink® Streptavidin R-Phycoerythrin (PROzyme). Other conjugated dye molecules are known to persons skilled in the art.

Labels include, but are not limited to: light-emitting, light-scattering, and light-absorbing compounds which generate or quench a detectable fluorescent, chemiluminescent, or bioluminescent signal (see, e.g., Kricka, L., Nonisotopic DNA Probe Techniques, Academic Press, San Diego (1992) and Garman A., Non-Radioactive Labeling, Academic Press (1997).). Fluorescent reporter dyes useful as labels include, but are not limited to, fluoresceins (see, e.g., U.S. Pat. Nos. 5,188,934, 6,008,379, and 6,020,481), rhodamines (see, e.g., U.S. Pat. Nos. 5,366,860, 5,847,162, 5,936,087, 6,051,719, and 6,191,278), benzophenoxazines (see, e.g., U.S. Pat. No. 6,140,500), energy-transfer fluorescent dyes, comprising pairs of donors and acceptors (see, e.g., U.S. Pat. Nos. 5,863,727; 5,800,996; and 5,945,526), and cyanines (see, e.g., WO 9745539), lissamine, phycoerythrin, Cy2, Cy3, Cy3.5, Cy5, Cy5.5, Cy7, Fluor X (Amersham), Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, Tetramethylrhodamine, and/or Texas Red, as well as any other fluorescent moiety capable of generating a detectable signal. Examples of fluorescein dyes include, but are not limited to, 6-carboxyfluorescein; 2′,4′,1,4,-tetrachlorofluorescein, and 2′,4′,5′,7′,1,4-hexachlorofluorescein. In certain aspects, the fluorescent label is selected from SYBR-Green, 6-carboxyfluorescein (“FAM”), TET, ROX, VICTM, and JOE. For example, in certain embodiments, labels are different fluorophores capable of emitting light at different, spectrally-resolvable wavelengths (e.g., 4-differently colored fluorophores); certain such labeled probes are known in the art and described above, and in U.S. Pat. No. 6,140,054. A dual labeled fluorescent probe that includes a reporter fluorophore and a quencher fluorophore is used in some embodiments. It will be appreciated that pairs of fluorophores are chosen that have distinct emission spectra so that they can be easily distinguished.

In still a further aspect, labels are hybridization-stabilizing moieties which serve to enhance, stabilize, or influence hybridization of duplexes, e.g., intercalators and intercalating dyes (including, but not limited to, ethidium bromide and SYBR-Green), minor-groove binders, and cross-linking functional groups (see, e.g., Blackburn et al., eds. “DNA and RNA Structure” in Nucleic Acids in Chemistry and Biology (1996)).

In further aspects, methods relying on hybridization and/or ligation to quantify miRNAs or cDNAs may be used, including oligonucleotide ligation (OLA) methods and methods that allow a distinguishable probe that hybridizes to the target nucleic acid sequence to be separated from an unbound probe. As an example, HARP-like probes, as disclosed in U.S. Publication No. 2006/0078894 may be used to measure the quantity of miRNAs. In such methods, after hybridization between a probe and the targeted nucleic acid, the probe is modified to distinguish the hybridized probe from the unhybridized probe. Thereafter, the probe may be amplified and/or detected. In general, a probe inactivation region comprises a subset of nucleotides within the target hybridization region of the probe. To reduce or prevent amplification or detection of a HARP probe that is not hybridized to its target nucleic acid, and thus allow detection of the target nucleic acid, a post-hybridization probe inactivation step is carried out using an agent which is able to distinguish between a HARP probe that is hybridized to its targeted nucleic acid sequence and the corresponding unhybridized HARP probe. The agent is able to inactivate or modify the unhybridized HARP probe such that it cannot be amplified.

In an additional embodiment of the method, a probe ligation reaction may be used to quantify miRNAs or cDNAs. In a Multiplex Ligation-dependent Probe Amplification (MLPA) technique (Schouten et al., Nucleic Acids Research 30:e57 (2002)), pairs of probes which hybridize immediately adjacent to each other on the target nucleic acid are ligated to each other only in the presence of the target nucleic acid. In some aspects, MLPA probes have flanking PCR primer binding sites. MLPA probes can only be amplified if they have been ligated, thus allowing for detection and quantification of miRNA biomarkers.

### 5. DIAGNOSING PD

In certain aspects, the invention provides various methods to diagnose PD. In some embodiments, these methods utilize assays to detect one or more of the above-identified miRNA biomarkers. Any assay that will detect one or more of the inventive miRNA biomarkers can be used, whether assayed individually or in combination by, e.g., high throughput methods such as q-PCR or qRT-PCR. For example, the level of a PD-specific miRNA in a biological sample can be detected, and a different level of that PD-specific miRNA biomarker in the biological sample as compared to a statistically validated threshold for that PD-specific miRNA indicates PD. That is, based on this comparison, a determination is made that the patient from whom the biological sample was taken has PD, is at a certain stage of PD, or is likely to develop PD.

Similarly, the levels of more than one (e.g., a pair, one pair and one single miRNA, two or more pairs and/or single miRNA) PD-specific miRNA in a biological sample can be detected, and different levels of those specific miRNAs in the biological sample, as compared to a statistically validated threshold for each of those specific miRNAs, indicates PD. That is, based on this comparison, a determination is made that the patient from whom the biological sample was taken has PD, is at a certain stage of PD, or is likely to develop PD.

The statistically validated threshold for a specific miRNA may be based upon the level of miRNA from a control population, e.g., healthy individuals or individuals at different stages of PD. Various control populations are otherwise described herein and in the Examples. The statistically validated threshold is related to the values used to characterize the level of the miRNA in the biological sample obtained from the subject or patient. Thus, if the miRNA level is an absolute value, then the control value is also based upon an absolute value. The statistically validated threshold can take a variety of forms. For example, a statistically validated threshold can be a single cut-off value, such as a median or mean. Or, a statistically validated threshold can be divided equally (or unequally) into groups, such as low, medium, and high groups, the low group being individuals least likely to have PD and the high group being individuals most likely to have PD. In some aspects, a threshold may be a range of values; miRNA concentrations or amounts falling within this value may be labeled as “inconclusive”; values above or below this threshold range may be characteristic of the presence or absence of PD.

Statistically validated thresholds, e.g., mean levels, median levels, or “cut-off” levels, may be established by assaying a large sample of individuals in the select population and using a statistical model such as the predictive value method for selecting a positivity criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics, William and Wilkins, Harual Publishing Co. Malvern, Pa., which is specifically incorporated herein by reference. A “cut-off” value may be separately determined for each miRNA assayed. Statistically validated thresholds also may be determined according to the methods described in the Examples hereinbelow. The levels of the assayed miRNAs in the biological sample may be compared to single control values or to ranges of control values. In one embodiment, a level of a specific miRNA in a biological sample from a patient (e.g., a patient having or suspected of having PD) is present at a higher or lower level (i.e., at a different level) than in comparable control biological samples from patients who do not have PD when the miRNA level in the biological sample exceeds a threshold of one and one-half standard deviations above the mean of the concentration as compared to the comparable control biological samples. More preferably, a level of a specific miRNA in a biological sample from a patient (e.g., a patient having or suspected of having PD) is present at a higher or lower level (i.e., at a different level) than in comparable control biological samples from patients not having PD when the level of the specific miRNA in the biological sample exceeds a threshold of two standard deviations above the mean of the concentration as compared to the comparable control biological samples. Most preferably, a level of a specific miRNA in a biological sample from a patient (e.g., a patient having or suspected of having PD) is present at a higher or lower level (i.e., at a different level) than in comparable control biological samples from patients not having PD when the level of the specific miRNA in the biological sample exceeds a threshold of three standard deviations above the mean of the concentration as compared to the comparable control biological samples.

The differential expression of a particular biomarker indicating a diagnosis or prognosis for PD may be more than, e.g., 1,000,000×, 100,000×, 10,000×, 1000×, 10×, 5×, 2×, 1× a particular threshold, or less than, e.g., 0.5×, 0.1×, 0.01×, 0.001×, 0.0001×, 0.000001× a particular threshold.

If the level of the specific miRNA in a biological sample is present at a different level than a statistically validated threshold, then the patient is more prone to have PD than are individuals with levels comparable to the statistically validated threshold. The extent of the difference between the subject's level and a statistically validated threshold is also useful for characterizing the extent of the risk of the subject being stricken with PD in the future. Further, the extent of the difference between the subject's level and a statistically validated threshold is also useful for characterizing the extent of PD progression or remission in the subject and, thereby, determining which individuals would most greatly benefit from certain therapies. In those cases, where the statistically validated threshold ranges are divided into a plurality of groups, such as statistically validated threshold ranges for individuals at high risk, average risk and low risk, the comparison involves determining into which group the subject's level of the relevant risk predictor falls.

An advantage of the present invention is that it is a rapid, relatively inexpensive and non-invasive method for diagnosing and assessing the prognosis of individuals to develop PD, to have asymptomatic or early-stage PD, or to be symptomatic of PD. In some aspects, tests may be performed multiple times on the same subject to assess disease progress. Some aspects of the method may be used to detect or diagnose PD. Other aspects of the method may be used to detect early-stage PD, asymptomatic PD, or persons with a risk of developing PD. Some aspects the method may be used to give a prognosis for PD in an individual. In some aspects, the efficacy of preventative or ameliorative treatments for PD may be evaluated, by assessing the expression of PD-specific miRNA biomarkers.

Finally, in some aspects, miRNA biomarker expression is used to determine the efficacy of treatment received by a patient for PD, that is, the miRNA levels of the patient may be assessed before treatment, and on one or more occasions after the administration of a treatment, to determine whether the treatment is effective.

Diagnoses and prognoses of the present invention may also be used in combination with evaluation of external motor and non-motor symptoms of PD, as well as other bioassays for PD, such as protein assays, or genomic testing.

Diagnoses and prognoses of the present invention may be used as part of the treatment of PD, i.e., to indicate the initiation of one or more PD therapies, discontinuation of one or more therapies, or an adjustment to one or more therapies (e.g., an increase or decrease to drug therapy, physical therapy and the like.).

In some aspects, diagnoses and prognoses of the present invention may be used as part of the treatment of PD-plus diseases, i.e., to indicate the initiation of one or more PD-plus therapies, discontinuation of one or more therapies, or an adjustment to one or more therapies (e.g., an increase or decrease to drug therapy, physical therapy and the like.).

Drug therapies include the administration of dopaminergic drugs (e.g., dopamine-replenishing or dopamine mimicking) including levodopa treatments, levodopa, or levodopa combination treatments, which may include, administration with carbodopa, a dopamine enhancer, and/or the COMT-inhibitor entacapone; dopamine agonists, including ropinirole, pramipexole, rotigotine, apomorphine, pergolide and bromocriptine; MAO-B inhibitors, which are used alone or with levodopa, including selegiline, rasagiline, zydis selegiline HCl salt; COMT-inhibitors used to boost the effects of levodopa include entacapone, tolcapone; amantadine; anti-cholinergenics such as trihexyphenidyl, and benztropine; antiglutamatergics such as memantine, safinomide; neurturin therapies; anti-apoptotics, such as omigapil and CEP-1347; promitochondrials, such as CoQ (Coenzyme Q10) and creatine; calcium channel blockers, including isradipine, and growth factors such as GDNF; as well as drugs or vaccines targeting alpha-synuclein.

Surgical therapies that may be indicated may include deep brain stimulation (DBS), involving implantation of a battery-powered electrode in the brain; operations directly on neural tissue including, thalamotomy; pallidotomy; subthalmatomy; dopamergic cell transplant

Physical therapies may include diet and exercise regimens. Complementary or alternative therapies may include administration of herbal supplements alone or in combination with one of the above drug or surgical therapies. Supplements may include antioxidants such as vitamins C and E, calcium, ginger root, green tea and green tea extracts, St John's Wort, Ginkgo biloba, milk thistle, vitamin B12, and folic acid.

In response to the diagnosis of PD, in some aspects of the method, a subject may be treated with one or more of PD treatments (e.g., a drug, exercise regime), or treated with a modification of an existing treatment, modified in response to the diagnosis or prognosis of PD in that subject.

The invention further encompasses kits useful for diagnosis or prognosis of PD in a subject, wherein the kits may comprise at least one oligonucleotide cDNA primer capable of hybridizing to one or more miRNA described above, wherein the oligonucleotide primer is complementary to one or more of the miRNA described above.

In some embodiments one or more miRNA may be selected from the group consisting of miR-505, miR-626, miR-222, miR-1826, miR-450b-3p, miR-1307, miR-647, miR-548b-3p, miR-192*, miR-506, miR-572, miR-671-5p, miR-9*, miR-1225-5p, miR-632, miR-99a*, miR-891b, miR-579, miR-708*, miR-1253, miR-200a, miR-455-3p, miR-192*, miR-485-5p, miR-488, and miR-518c.

In some embodiments, the miRNAs are selected from the group consisting of miR-505, miR-1307, miR-647, miR-548b-3p, miR-192*, miR-506, miR-626, miR-1826, miR-572, miR-671-5p, miR-222, miR-9*, and miR-1225-5p; from the group consisting of the miRNA biomarker pairs miR-1826/miR-450b-3p, miR-1307/miR-632, miR-647/miR-99a*, miR-1225-5p/miR-891b, miR-579/miR-708*, miR-506/miR-1253, miR-200a/miR-455-3p, miR-192*/miR-485-5p, and miR-488/miR-518c*; from the group consisting of the miRNA pairs miR-1826/miR-450b-3p, miR-506/miR-1253, miR-200a/miR-455-3p, miR-192*/miR-485-5p, and miR-488/miR-518c*; from the group consisting of miR-505, miR-626, and miR-222; from the group consisting of biomarker pairs miR-1826/miR-450b-3p, miR-506/miR-1253, miR-200a/miR-455-3p, miR-192*/miR-485-5p, and miR-488/miR-518c*, and miR-505, miR-626, miR-222. In some embodiments of the kit, the miRNA is miR-505. In some embodiments of the kit, the miRNA are the biomarker pair miR-1826/miR-450b-3p. In other embodiments, the miRNA are miR505, miR-1826, miR-450b-3p, and miR-626.

In some embodiments, kits may include suitable reagents for extracting miRNA from a sample; amplifying the amount of miRNA or complements thereof in sample (e.g, PCR reagents); suitable reagents (e.g., PCR reagents) or devices (microarray chips and the like) for determining the concentration of miRNA in a sample.

Having now generally described the invention, the same will be more readily understood through reference to the following examples, which are provided by way of illustration, and are not intended to be limiting of the present invention, unless specified.

### EXAMPLES

The following examples illustrate various embodiments of the invention and are not intended to limit the scope of the invention.

### Example 1

### Materials and Methods

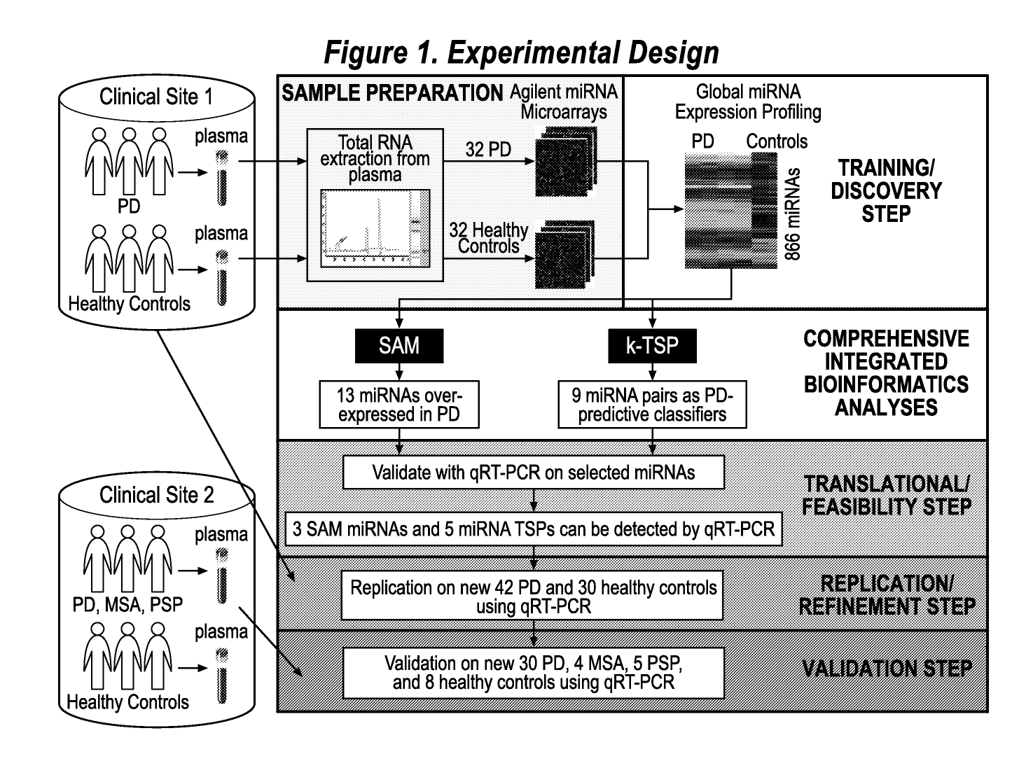

The experimental design for this study is shown in FIG. 1. Plasma samples of healthy controls and PD patients were obtained from the St. Mary's Health Care Hauenstein Parkinson's Center in Grand Rapids, Mich. In addition, PD, PSP, MSA patients and healthy controls recruited from the Department of Neurology at Umeå University Hospital (UUH) were used as a new, independent validation set. All subjects provided their informed consent and this study was approved by the institutional review boards of Saint Mary's Health Care, Umeå University Hospital, and the Van Andel Research Institute. PD patients were evaluated according to the UK PD Diagnostic Criteria with Hoehn and Yahr staging. Peripheral blood samples were obtained using 10 ml EDTA tubes, placed on ice immediately and centrifuged at 4° C., 1,000 g for 15 minutes. Plasma supernatant was aliquotted into 500 μl aliquots and stored immediately at −80° C. until analysis. Patient characteristics are shown in Table 2.

TABLE 2
Characteristics of PD and normal control samples

| | | |
|---|---|---|
| Characteristics | Parkinson's Disease | Normal Control |

Discovery set (miRNA microarray)

| | | | | |
|---|---|---|---|---|
| Sex | Male | Female | Male | Female |
| N | 16 (50%) | 16 (50%) | 15 (47%) | 17 (53%) |
| Age, year | | | | |
| Median | 65 | 69 | 67 | 68 |
| Mean | 66 ± 11 | 67 ± 11 | 65 ± 10 | 62 ± 17 |
| Hoehn and Yahr | 1.75 ± 2.25 | 1.50 ± 2.39 | | |
| Rating | | | | |

Replication set (qRT-PCR)

| | | | | |
|---|---|---|---|---|
| Sex | Male | Female | Male | Female |
| N | 20 (48%) | 22 (52%) | 10 (33%) | 20 (67%) |
| Age, year | | | | |
| Median | 69 | 73 | 65 | 63 |
| Mean | 68 ± 6 | 72 ± 8 | 64 ± 15 | 59 ± 14 |
| Hoehn and Yahr | 1.75 ± 1.83 | 1.63 ± 1.51 | | |
| Rating | | | | |

Validation set (qRT-PCR)

| | | | | |
|---|---|---|---|---|
| Sex | Male | Female | Male | Female |
| N | 16 (53%) | 14 (47%) | 3 (37.5%) | 5 (62.5%) |
| Age, year | | | | |
| Median | 66 | 73 | 71 | 73 |
| Mean | 68 ± 10 | 71 ± 7 | 71 ± 3 | 73 ± 4 |
| Hoehn and Yahr | 2.06 ± 0.66 | 2.38 ± 0.63 | | |
| Rating | | | | |

Total RNA with small RNAs, including miRNAs, was extracted using the TRI reagent RT-blood protocol (Molecular Research Center) with slight modification. Polyacryl carrier was added to improve RNA recovery. FirstChoice human brain reference RNA (Ambion) was applied as a positive control. RNA samples were processed, labeled, and hybridized onto whole human genome miRNA microarrays v.3 containing 866 human miRNAs (Agilent). Microarray slides were scanned using the Agilent G3 High Resolution Scanner and the resultant data were extracted by Agilent Feature Extraction software v.10.7.3.1. Microarray data was background-corrected and normalized before statistical analysis.

The K-TSP (k-Top Scoring Pair) algorithm was applied to identify paired PD-predictive miRNA classifiers. This algorithm directly addresses the tradeoff between sample size and model complexity in machine learning algorithms as well as between “bias-variance” by incorporating simplifying assumptions. In brief, this comparison-based method seeks to discriminate the disease group from appropriate controls by finding pairs of miRNAs whose expression levels typically invert from one group to another. SAM (Significance Analysis of Microarrays) was used to identify differentially expressed single miRNAs between healthy controls and PD patients. The Newton's Gamma-Gamma Bernoulli correction [Newton Mass., Kendziorski C M, Richmond C S, Blattner F R, Tsui K W. On differential variability of expression ratios: improving statistical inference about gene expression changes from microarray data. J Comput Biol. 2001; 8(1):37-52.] was applied to both analyses to provide more precise estimates of differentially-expressed miRNAs and more accurate assessments of significant changes.

TargetScanHuman (www.targetscan.org) which predicts biological targets of miRNAs by searching for the presence of conserved 7mer and 8mer sites that match the seed region of each miRNA was used to search for predicted miRNA targets of the classifiers.

QRT-PCR using TaqMan miRNA-specific assays were performed in triplicate on a new cohort of samples to determine the sensitivity and specificity of PD-predictive classifiers (PD biomarkers) identified through K-TSP and SAM. In brief, 40 ng of total RNA was reverse transcribed to cDNA in a PCR thermal cycler using the Taqman miRNA-specific assay and qRT-PCR was performed using the StepOnePlus real time PCR system (Applied Biosystems, Foster City, Calif.) to detect each miRNA expression. Human miR-16 and non-human ath-miR-156a were used as an endogenous and negative control, respectively. To calculate the expression of each target miRNA relative to each endogenous control, the comparative C_{T} (ΔΔC_{T}) method was used [Livak K J, Schmittgen T D. Analysis of relative gene expression data using real-time quantitative PCR and the 2(-Delta Delta C(T)) Method. Methods. 2001 December, 25(4):402-8]. Each candidate miRNA biomarker whose qRT-PCR expression was in concordance with its microarray expression was then performance evaluated using a new set of PD patients and normal controls. 95% normalized C_{T} values of individual miRNAs were used as thresholds for classifying PD and normal controls. Multiple thresholds approach was applied to overcome the potential inter-center variability. [Hu W T, Chen-Plotkin A, Arnold S E, et al. Biomarker discovery for Alzheimer's disease, frontotemporal lobar degeneration, and Parkinson's disease. Acta Neuropathol. 2010 September; 120(3):385-99.]

Using samples from SMHCPC, 32 treated PD patients were assigned as the discovery set (microarray) and 42 patients as the replication set (qRT-PCR). Sixty-two normal controls were also included. Normal control spouses of PD patients were used to minimize the environmental component as a confounding factor of this study. Data was analyzed in paired PD-control spouse fashion. In addition, 20 treated PD, 10 newly diagnosed untreated PD, 5 PSP, 4 MSA, and 8 healthy controls from UUH were used as a new, independent validation set (qRT-PCR). The clinical characteristics were similar among discovery, replication, and validation sets (Table 2).

### Example 2

### Brain- and Plasma-Specific miRNAs can be Detected in Plasma Using miRNA Microarrays

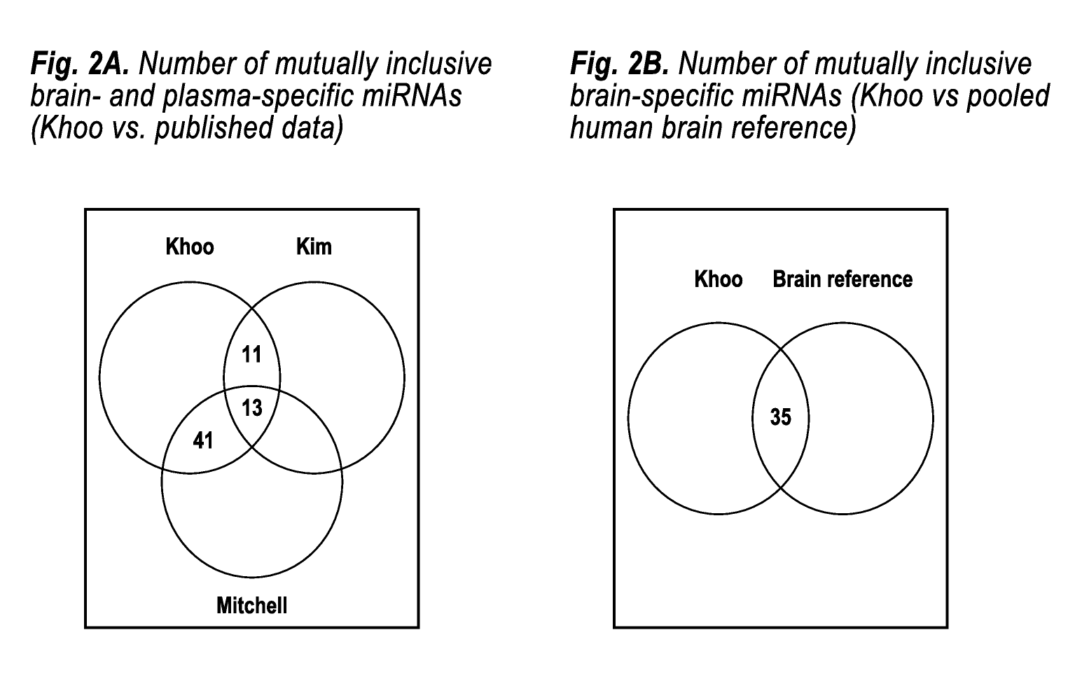

In a preliminary study, 24 brain-specific miRNAs that were reported by Kim et al. [(2007) A MicroRNA feedback circuit in midbrain dopamine neurons. Science 317:1220-1224] and 54 plasma-specific miRNAs cited by Mitchell et al. [(2008) Circulating microRNAs as stable blood-based markers for cancer detection. Proc. Natl. Acad. Sci. (USA) 105:10513-10518] were detected in our healthy control plasma (FIG. 2A). In addition, 35 miRNAs in the healthy control plasma that were also expressed in the commercially available human brain tissue reference were also detected (FIG. 2B). These results showed that it is feasible to detect brain- and plasma-specific circulating miRNAs in plasma.

### Example 3

### K-TSP Defined 9 Pairs of PD Predictive miRNA Classifiers

Using the novel k-TSP algorithm [Tan, A C et al. (2005) Simple decision rules for classifying human cancers from gene expression profiles. Bioinformatics 21:3896-3904], the use of which has proven feasible to handle small sample learning problems and generate accurate classifiers [Rajeshkumar N V, et al. (2009) Antitumor effects and biomarkers of activity of AZD0530, a Src inhibitor, in pancreatic cancer. Clin Cancer Res 15:4138-4146; Pitts T M, et al (2010) Development of an integrated genomic classifier for a novel agent in colorectal cancer: approach to individualized therapy in early development. Clin Cancer Res 16: 3193-3204], 9 pairs of PD-predictive miRNA biomarkers were identified from the microarray data.

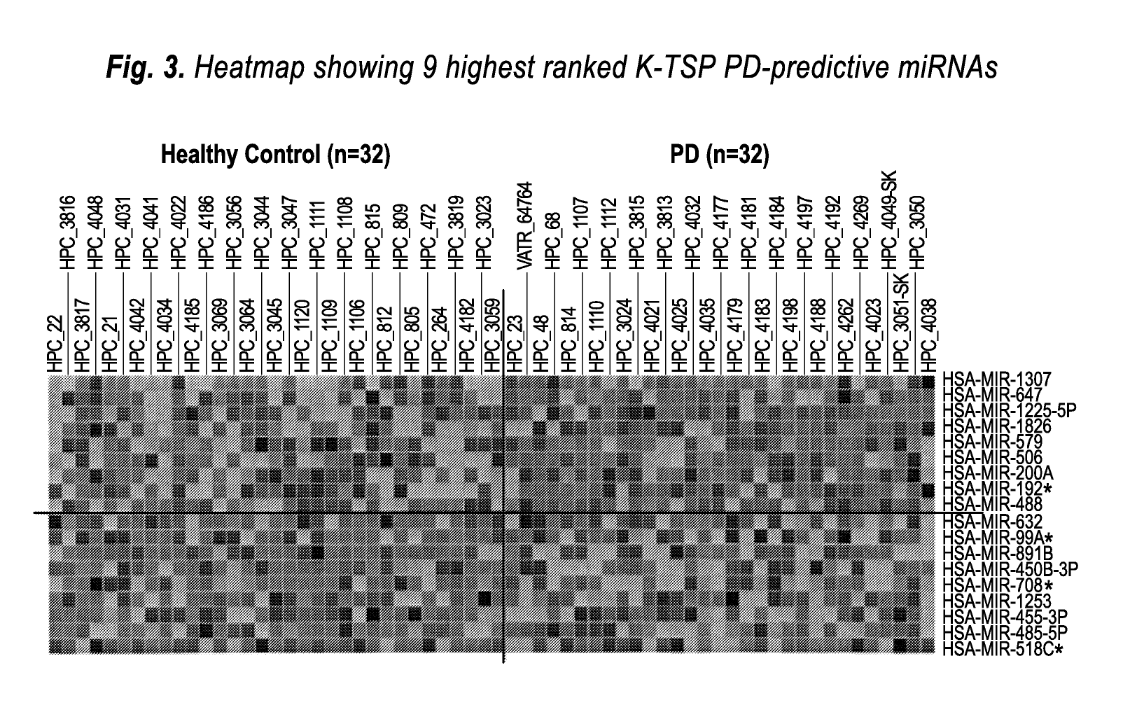

The 9 paired classifiers were miR-1307/miR-632, miR-647/miR-99a*, miR-1225-5p/miR-891b, miR-1826/miR-450b-3p, miR-579/miR-708*, miR-506/miR-1253, miR-200a/miR-455-3p, miR-192*/miR-485-5p, and miR-488/miR-518c*. The interpretation for the first miR-1307/miR-632 is: if the expression of miR-1307 is higher than miR-632, then the patient is predicted as PD, else it is predicted as normal control. Similar interpretations are also applied to other miRNA pairs. As each individual miRNA pair can be used as a classifier, the final prediction is based on the majority voting of the 9 miRNA pairs. Microarray expression patterns for these pairs are shown in FIG. 3. Leave-one-out cross-validation accuracy is 58%, sensitivity is 71% and specificity is 50% for this training set (32 PD patients and 32 healthy controls).

Mean fold changes in the microarray experiments are provided between Parkinson's disease patients and healthy controls (HC) in Table 3, for each member of the pair. For example, the miRNA biomarker HSA-MIR-1307 showed a 1.67-fold higher expression in samples from PD patients as opposed to HC; the second member of the pair, hsa-mir-632, showed a 0.70-fold expression (i.e., a lower expression) in samples from PD patients as opposed to HC.

TABLE 3
Mean fold changes in K-TSP pairs between
PD patients and healthy controls.

| | Fold Change | | Fold Change |
|---|---|---|---|
| Pair member 1 | (PD vs. HC) | Pair member 2 | (PD vs. HC) |
| hsa-miR-1307 | 1.67 | hsa-miR-632 | 0.70 |
| hsa-miR-647 | 1.50 | hsa-miR-99a* | 0.75 |
| hsa-miR-1225-5p | 1.35 | hsa-miR-891b | 0.70 |
| hsa-miR-1826 | 1.43 | hsa-miR-450b-3p | 0.77 |
| hsa-miR-579 | 1.22 | hsa-miR-708* | 0.64 |
| hsa-miR-506 | 1.38 | hsa-miR-1253 | 0.81 |
| hsa-miR-200a | 1.36 | hsa-miR-455-3p | 0.80 |
| hsa-miR-192* | 1.46 | hsa-miR-485-5p | 0.76 |
| hsa-miR-488 | 1.13 | hsa-miR-518c* | 0.62 |

### Example 4

### SAM Identified 13 Most-Differentially Expressed miRNAs

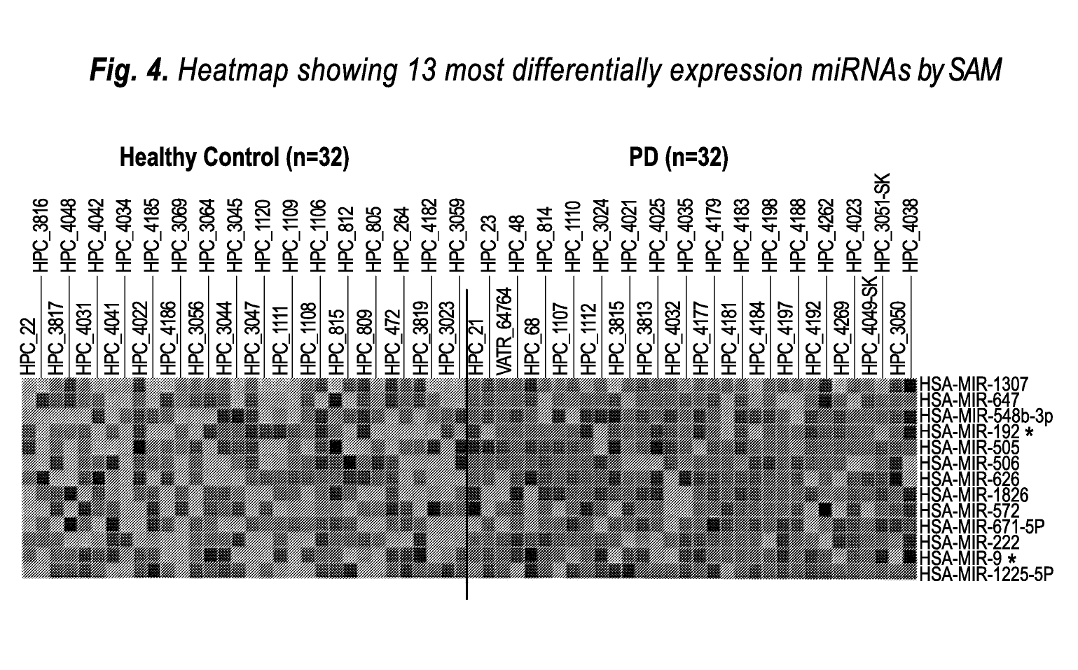

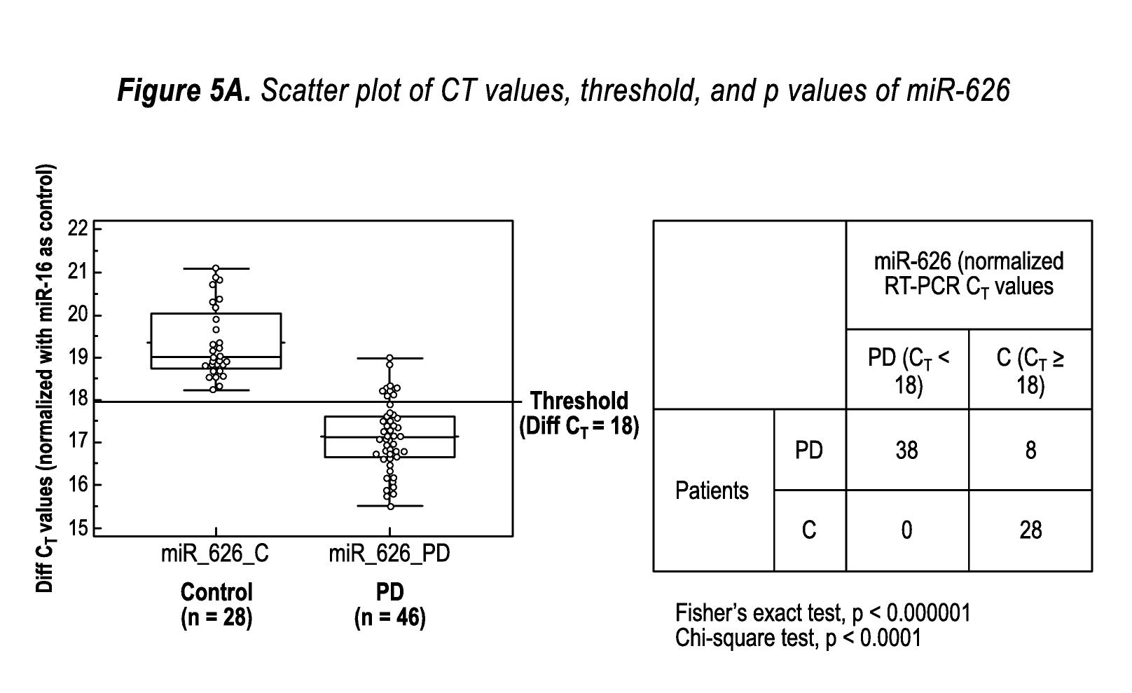

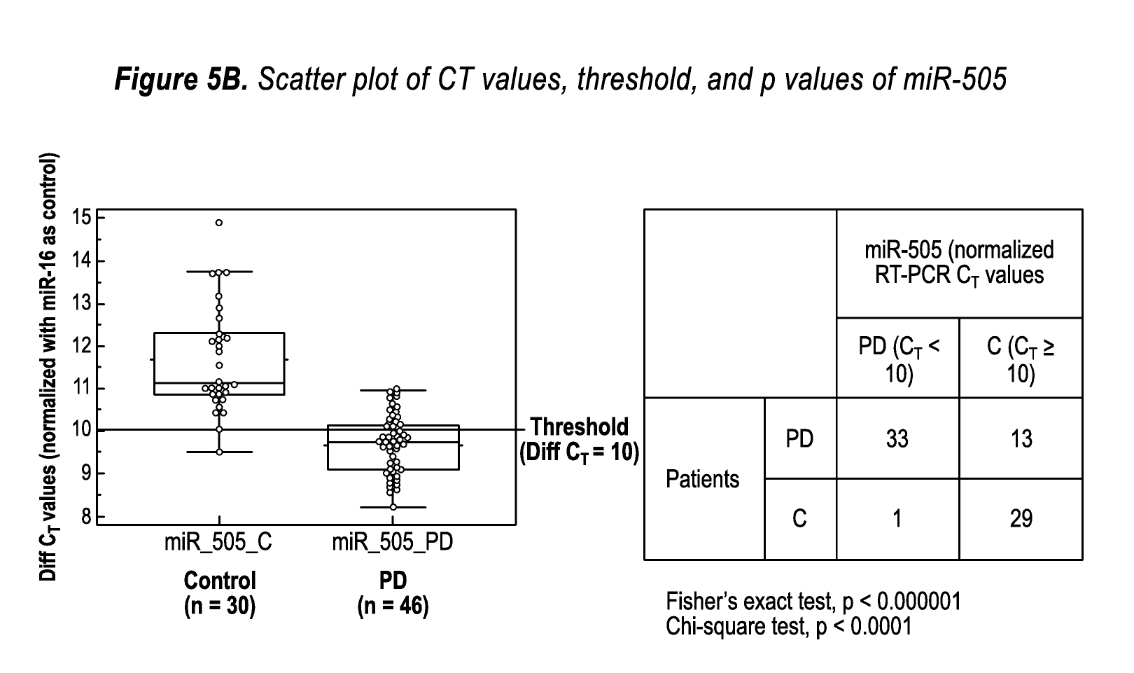

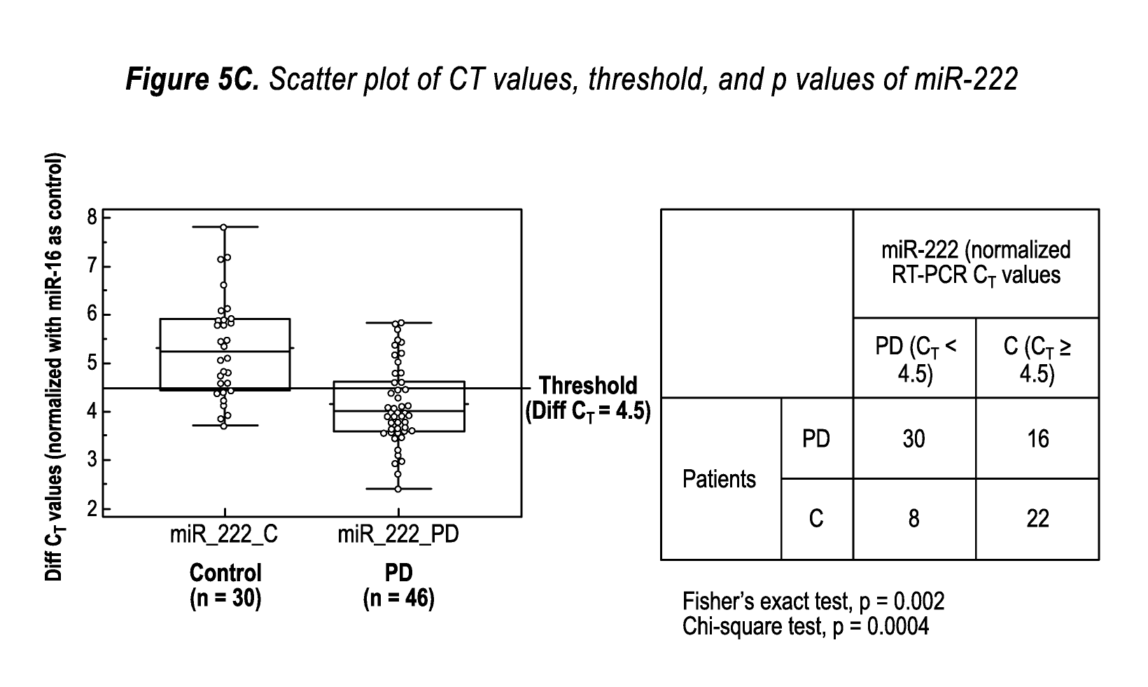

SAM analysis with 500 permutations was applied to identify differentially expressed miRNAs between PD and healthy control samples. The thirteen most-differentially expressed miRNAs were identified: miR-1307, miR-647, miR-548b-3p, miR-192*, miR-505, miR-506, miR-626, miR-1826, miR-572, miR-671-5p, miR-222, miR-9*, and miR-1225-5p. Microarray expression patterns for the miRNAs identified with SAM are shown in FIG. 4. Six miRNAs (miR-1307, miR-647, miR-192*, miR-506, miR-1826, and miR-1225-5p) were mutually inclusive with the PD-predictive classifiers. Interestingly, miR-626 is a possible target for both of the PD-related genes LRRK2 and PARK2, while miR-222 and miR-505 are possible targets for PARK2. Mean fold changes in the microarray experiments are provided between Parkinson's disease patients and healthy controls (HC) in Table 4, below.

TABLE 4
Mean fold changes in SAM microarray experiments
between PD patients and healthy controls.

| | Fold Change |
|---|---|
| miRNA biomarker | (PD vs HC) |
| hsa-miR-1307 | 1.77 |
| hsa-miR-647 | 1.58 |
| hsa-miR-548b-3p | 1.48 |
| hsa-miR-192* | 1.46 |
| hsa-miR-505 | 1.45 |
| hsa-miR-506 | 1.46 |
| hsa-miR-626 | 1.38 |
| hsa-miR-1826 | 1.44 |
| hsa-miR-572 | 1.53 |
| hsa-miR-671-5p | 1.63 |
| hsa-miR-222 | 1.53 |
| hsa-miR-9* | 1.37 |
| hsa-miR-1225-5p | 1.32 |

### Example 5

### MiRNA Expression can be Validated Using qRT-PCR

The miRNA expressions of all 9 pairs of PD-classifiers as well as 3 of the most differentially expressed miRNAs: miR-626, miR-222, and miR-505, was validated using qRT-PCR, on 16 PD samples and a pooled healthy control (n=39). A very high concordance (>70%) was observed between miRNAs expressions measured in the microarrays vs. qRT-PCR. However, 4 top scoring pairs (TSPs) were not detectable by qRT-PCR even after optimizing with various cycles and RNA quantities. Thus, only 5 k-TSP classifiers (miR-1826/miR-450b-3p-TSP1, miR-506/miR-1253-TSP2, miR-200a/miR-455-3p-TSP3, miR-192*/miR-485-5p-TSP4, and miR-488/miR-518c*-TSP5) and 3 differentially expressed miRNAs (miR-626, miR-222, and miR-505) were evaluated further for biomarker sensitivity and specificity.

### Example 6

### K-TSP PD-Predictive miRNAs Showed High Sensitivity

RNA from 42 PD subjects and 30 normal controls from a new, independent cohort and 4 PD samples from the training set were used in miRNA qRT-PCR. K-TSP pairs were given abbreviations as follows: miR-1826/miR-450b-3p (TSP1), miR-506/miR-1253 (TSP2), miR-200a/miR-455-3p (TSP3), miR-192*/miR-485-5p (TSP4), and miR-488/miR-518c* (TSP5). Sensitivity (% of PDs who test positive), specificity (% of normal controls who test negative), positive and negative predicted values of each TSP as well as various combinations of TSP were evaluated. The mean fold change data are provided in Table 5, below. The results are summarized in Table 6 below. TSP1 (miR-1826/miR-450b-3p pair) showed the highest predictive power with 100% sensitivity, 56% specificity, and 92% and 100% positive and negative predicted value, respectively. A combination of TSP1, TSP3, and TSP4 improved specificity (64%) while maintained high sensitivity (96%).

TABLE 5
Mean fold changes in miRNA biomarker pairs and single
miRNA between PD patients and healthy controls in RT-PCR.
KTSP pairs

| | | Fold Change | | Fold Change |
|---|---|---|---|---|
| Name | Pair member 1 | (PD vs. HC) | Pair member 2 | (PD vs. HC) |
| TSP1 | hsa-miR-1826 | 2.90 | hsa-miR-450b-3p | 1.76 |
| TSP2 | hsa-miR-506 | 1.36 | hsa-miR-1253 | 1.45 |
| TSP3 | hsa-miR-200a | 1.82 | hsa-miR-455-3p | 0.78 |
| TSP4 | hsa-miR-192* | 1.55 | hsa-miR-485-5p | 2.28 |
| TSP5 | hsa-miR-488 | N.D. | hsa-miR-518c* | 2.52 |

SAM single miRNA

| miRNA | FC (PD vs C) |
|---|---|
| hsa-miR-505 | 4.09 |
| hsa-miR-626 | 4.68 |
| hsa-miR-222 | 2.24 |

TABLE 6
Performance evaluation of k-TSP PD-predictive miRNA biomarkers show
high sensitivity and miRNA biomarkers by SAM show high specificity

| | | # | | | + | − |
|---|---|---|---|---|---|---|
| | | Healthy | | | Pre- | Pre- |
| | #PD | control | Sensi- | Speci- | dicted | dicted |
| miRNA | Detected | detected | tivity | ficity | Value | Value |
| TSP 1 | 45 | 9 | 100% | 56% | 92% | 100% |
| TSP 2 | 41 | 1 | 32% | 0% | 93% | 0% |
| TSP 3 | 46 | 25 | 96% | 9% | 66% | 50% |
| TSP 4 | 43 | 28 | 0% | 100% | 0% | 31% |
| TSP 5 | 33 | 5 | 0% | 20% | 0% | 3% |
| TSP 1-5 | 45 | 11 | 96% | 27% | 94% | 60% |
| TSP 1-4 | 45 | 11 | 96% | 55% | 90% | 75% |
| TSP 1-3 | 45 | 11 | 96% | 55% | 90% | 75% |
| TSP 1, 3, 4 | 45 | 11 | 96% | 64% | 91% | 78% |
| miR-626 | 46 | 28 | 83% | 100% | 100% | 78% |
| miR-505 | 46 | 30 | 72% | 97% | 97% | 69% |
| miR-222 | 46 | 30 | 78% | 73% | 79% | 58% |

Note:
+, positive;
−, negative.

An interesting observation was made regarding the number of healthy controls that could be detected using TSP miRNAs. Most controls (97%) could be detected using SAM miRNA candidates (FIG. 7) but only a handful (42%) could be detected by TSP miRNA classifiers, with detection power varying from 93% to as low as 3%. On the other hand, most TSP classifiers could be detected in PD samples (94%). Like the PD samples, each control sample was aliquotted from one original sample source for qRT-PCR. The same batch of miRNA qRT-PCR probes were also used for both PD and control samples. Thus, the variability in samples and probes could be ruled out since the same sample aliquots and miRNA probes were used in the qRT-PCR assays. Therefore, it is likely that certain miRNAs were so low in expression that they could barely be detected or completely undetectable in healthy controls using qRT-PCR, when compared with PD samples. These miRNAs have the potential of becoming PD-progression biomarkers (e.g., healthy control—very low or no expression but expression gradually increases with PD stage).

Interestingly, the miRNA expression of TSP pairs in 30%-93% of normal controls and in 89%-98% of PD samples (e.g. TSP 1: 9/30 in controls, 45/46 in PD; TSP 4: 28/30 in controls, 43/46 in PD) was detected. However, in the validation set, the expression of TSP pairs could not be detected (except in 1 PD and 1 healthy control) using qRT-PCR.

### Example 7

### Differentially Expressed miRNAs by SAM Showed High Specificity

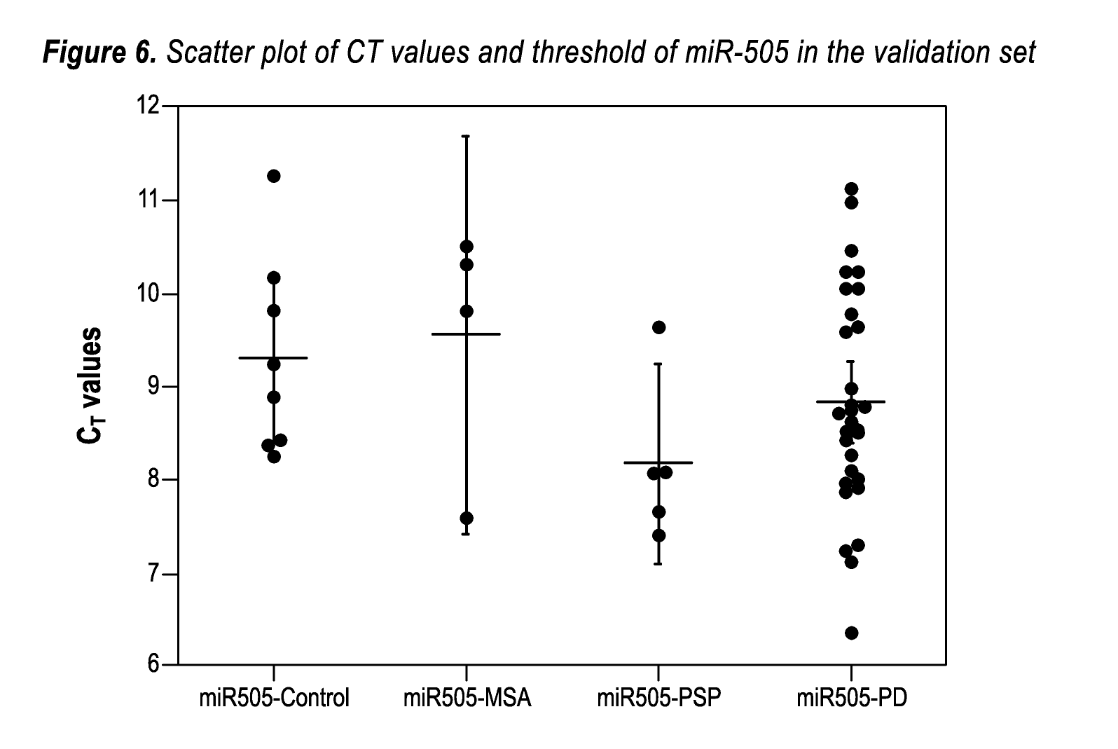

RNA from the same new independent cohort (42 PDs and 30 normal controls) and 4 PDs from the training set were used to evaluate the miRNA expression of miR-626, miR-505, and miR-222. Scatter plots of normalized C_{T} (cycle threshold, a relative measure of oligonucleotide concentration) values, thresholds (95% cut-off), and p values of each miRNA are shown in FIGS. 6A-6C. The biomarker miR-626 showed the highest predictive power: 83% sensitivity, 100% specificity, 100% positive predicted value, and 78% negative predicted value (see Table 6; FIG. 7).

In the validation set, miR-626 also showed high specificity (100%) but 0% sensitivity while miR-505 showed 76% sensitivity and 18% specificity (FIG. 6).

### Example 8

### The Combination of TSP1, miR-626, and miR-505 Showed 91% Sensitivity and 100% Specificity

Since TSP classifiers showed high sensitivity, while miR-626, miR-505, and miR-222 from SAM showed high specificity, we integrated both sets of data to evaluate their combined predictive power. We found the combination of TSP1, miR-626, and miR-505 achieved the highest predictive power: 91% sensitivity, 100% specificity, 100% positive predicted value, and 88% negative predicted value (See Table 7 below).

TABLE 7
Identification of plasma-based PD biomarker panels with high predictive
performance

| | | | | | + | − |
|---|---|---|---|---|---|---|
| | # | # healthy | | | Pre- | Pre- |
| | PD | controls | Sensi- | Speci- | dicted | dicted |
| miRNA | Detected | detected | tivity | ficity | Value | Value |
| TSP 1 + | 46 | 30 | 98% | 83% | 90% | 96% |
| miR626 | | | | | | |
| TSP 1 + | 46 | 30 | 100% | 87% | 92% | 95% |
| miR505 | | | | | | |
| TSP 1 + | 46 | 30 | 98% | 63% | 80% | 95% |
| miR-222 | | | | | | |
| TSP 1 + | 46 | 30 | 91% | 100% | 100% | 88% |
| miR-626 + | | | | | | |
| miR505 | | | | | | |

Note:
+, positive;
−, negative.

The performance of this specific panel of multiple biomarkers could not be evaluated in the validation set due to the undetectable expressions of TSP1. We also examined the associations between age, gender, and treatment of these biomarkers but there were no significant statistical differences between these groups.

1. A method of determining whether a subject has Parkinson's disease, is at increased risk of developing Parkinson's disease, or has Parkinson's disease that is progressing or is in remission, comprising:
obtaining a biological sample from the subject;
detecting the level of one or more miRNA in the biological sample, wherein the one or more miRNA are selected from the group consisting of miR-505, miR-626, miR-222, miR-1826, miR-450b-3p, miR-1307, miR-647, miR-548b-3p, miR-192*, miR-506, miR-572, miR-671-5p, miR-9*, miR-1225-5p, miR-632, miR-99a*, miR-891b, miR-579, miR-708*, miR-1253, miR-200a, miR-455-3p, miR-192*, miR-485-5p, miR-488, and miR-518c;
comparing a level of the one or more miRNA in the biological sample to a statistically validated threshold for each miRNA, which statistically validated threshold for each miRNA is based on the level of the miRNA in comparable control biological samples; and
determining that the subject has Parkinson's disease or is at increased risk of developing Parkinson's disease, or that the Parkinson's disease has progressed or is in remission in the subject, when the one or more miRNA are at a different level in the biological sample as compared to the statistically validated threshold for each miRNA.

2. The method of claim 1, wherein the detection step further comprises amplifying any of the one or more miRNA present in the sample, using at least one oligonucleotide primer, to create an amplification product unique to each miRNA, wherein the at least one oligonucleotide primer comprises a region complementary to the miRNA.

3. The method of claim 2, wherein the oligonucleotide primer comprises cDNA.

4. The method of claim 1, wherein the detection step further comprises using an oligonucleotide having a sequence complementary to the miRNA sequence.

5. The method of claim 4, wherein the oligonucleotide comprises cDNA.

6. The method of claim 4, wherein the oligonucleotide is a primer.

7. The method of claim 2, wherein the amplification product is a cDNA.

8. The method of claim 7, wherein the amplification product is a cDNA having a sequence complementary to any miRNA present in the sample.

9. The method of claim 2, wherein the amplification step further comprises adding RNA bases to one or more ends of the miRNA oligonucleotide.

10. The method of claim 1, wherein the detection step further comprises using a probe to detect the presence of any of the miRNA in the biological sample.

11. The method of claim 10, wherein the probe is tagged with a detection signal.

12. The method of claim 11, wherein the probe comprises an oligonucleotide having a sequence that is complementary or identical to all or a region of the miRNA sequence.

13. The method of claim 12, wherein the probe is cDNA.

14. The method of claim 1, wherein the biological sample from the subject is selected from brain tissue, cerebrospinal fluid, blood plasma, or a combination thereof.

15. The method of claim 14, wherein the biological sample from the subject is blood plasma.

16. The method of claim 1, wherein the comparable control biological samples are from healthy subjects.

17. The method of claim 1, wherein the comparable control biological samples are from subjects having Parkinson's disease.

18. The method of claim 1, wherein the biological sample is compared to comparable control biological samples from subjects with Parkinson's disease and comparable control biological samples from healthy subjects.

19. The method of claim 1, wherein it is determined from the determining step that the subject has Parkinson's disease or is at increased risk of developing Parkinson's disease, or that the Parkinson's disease has progressed or is in remission in the subject initiates a treatment for Parkinson's disease.

20. The method of claim 19, further comprising treating the subject with a new or modified treatment for Parkinson's disease.

21. The method of claim 1, wherein the one or more miRNA are selected from miR-505, miR-626, miR-1307, miR-647, miR-548b-3p, miR-192*, miR-506, miR-1826, miR-572, miR-671-5p, miR-222, miR-9*, and miR-1225-5p.

22. The method of claim 1, wherein two or more miRNA are selected from one or more of the biomarker pairs: miR-1826/miR-450b-3p, miR-1307/miR-632, miR-647/miR-99a*, miR-1225-5p/miR-891b, miR-579/miR-708*, miR-506/miR-1253, miR-200a/miR-455-3p, miR-192*/miR-485-5p, and miR-488/miR-518c*.

23. The method of claim 22, wherein the two or more miRNA are selected from one or more of the biomarker pairs miR-1826/miR-450b-3p, miR-506/miR-1253, miR-200a/miR-455-3p, miR-192*/miR-485-5p, and miR-488/miR-518c*.

24. The method of claim 21, wherein the one or more miRNA are selected from one or more of miR-505, miR-626, and miR-222.

25. The method of claim 1, wherein the one or more miRNA are selected from: one or more of the biomarker pairs miR-1826/miR-450b-3p, miR-506/miR-1253, miR-200a/miR-455-3p, miR-192*/miR-485-5p, and miR-488/miR-518c*, and/or single miRNA miR-505, miR-626, and miR-222.

26. The method of claim 25, wherein the miRNA is miR-505.

27. The method of claim 25, wherein the miRNA are the biomarker pair miR-1826/miR-450b-3p.

28. The method of claim 22, wherein the miRNA are miR-50, miR-1826, miR-450b-3p, and miR-626.

29. A method of treating Parkinson's disease, comprising
obtaining a biological sample from a subject;
detecting the level of one or more miRNA in the biological sample, wherein the miRNA is selected from the group consisting of miR-505, miR-626, miR-222, miR-1826, miR-450b-3p, miR-1307, miR-647, miR-548b-3p, miR-192*, miR-506, miR-572, miR-671-5p, miR-9*, miR-1225-5p, miR-632, miR-99a*, miR-891b, miR-579, miR-708*, miR-1253, miR-200a, miR-455-3p, miR-192*, miR-485-5p, miR-488, and miR-518c;
comparing a level of the miRNA in the biological sample to a statistically validated threshold for the miRNA, which statistically validated threshold for the miRNA is based on the level of the miRNA in comparable control biological samples;
determining that the subject has Parkinson's disease or is at increased risk of developing Parkinson's disease, or that the Parkinson's disease has progressed or is in remission in the subject, when the miRNA is at a different level in the biological sample as compared to the statistically validated threshold for the miRNA,
wherein it is determined from the determining step that the subject has Parkinson's disease or is at increased risk of developing Parkinson's disease, or that the Parkinson's disease has progressed or is in remission in the subject initiates a treatment for Parkinson's disease; and
treating the subject with a new or modified treatment for Parkinson's disease.

30. A kit for determining whether a subject has Parkinson's disease, is at increased risk of developing Parkinson's disease, or has Parkinson's disease that is progressing or is in remission, comprising one or more oligonucleotide primers capable of hybridizing to one or more miRNA, wherein the one or more oligonucleotide primer is complementary to one or more miRNA selected from the group consisting of miR-505, miR-626, miR-222, miR-1826, miR-450b-3p, miR-1307, miR-647, miR-548b-3p, miR-192*, miR-506, miR-572, miR-671-5p, miR-9*, miR-1225-5p, miR-632, miR-99a*, miR-891b, miR-579, miR-708*, miR-1253, miR-200a, miR-455-3p, miR-192*, miR-485-5p, miR-488, and miR-518c.

31. The kit of claim 30, wherein the miRNAs are selected from the group consisting of miR-505, miR-1307, miR-647, miR-548b-3p, miR-192*, miR-506, miR-626, miR-1826, miR-572, miR-671-5p, miR-222, miR-9*, and miR-1225-5p.

32. The kit of claim 30, wherein the miRNA are selected from the group consisting of the miRNA biomarker pairs miR-1826/miR-450b-3p, miR-1307/miR-632, miR-647/miR-99a*, miR-1225-5p/miR-891b, miR-579/miR-708*, miR-506/miR-1253, miR-200a/miR-455-3p, miR-192*/miR-485-5p, and miR-488/miR-518c*.

33. The kit of claim 30, wherein the miRNA are selected from the group consisting of the miRNA pairs miR-1826/miR-450b-3p, miR-506/miR-1253, miR-200a/miR-455-3p, miR-192*/miR-485-5p, and miR-488/miR-518c*.

34. The kit of claim 30, wherein the miRNA are selected from the group consisting of miR-505, miR-626, and miR-222.

35. The kit of claim 30, wherein the miRNA are selected from the group consisting of biomarker pairs miR-1826/miR-450b-3p, miR-506/miR-1253, miR-200a/miR-455-3p, miR-192*/miR-485-5p, and miR-488/miR-518c*, and miR-505, miR-626, miR-222.

36. The kit of claim 30, wherein the miRNA is miR-505.

37. The kit of claim 30, wherein the miRNA are the biomarker pair miR-1826/miR-450b-3p.

38. The kit of claim 30, wherein the miRNA are miR505, miR-1826, miR-450b-3p, and miR-626.


SEQUENCE LISTING (26 sequences)

SEQ ID NO: 1 - RNA, 22 nt, Homo sapiens
acucggcguggcgucggucgug

SEQ ID NO: 2 - RNA, 19 nt, Homo sapiens
gugucugcuuccuguggga

SEQ ID NO: 3 - RNA, 21 nt, Homo sapiens
guggcugcacucacuuccuuc

SEQ ID NO: 4 - RNA, 22 nt, Homo sapiens
caagcucgcuucuaugggucug

SEQ ID NO: 5 - RNA, 22 nt, Homo sapiens
guggguacggcccagugggggg

SEQ ID NO: 6 - RNA, 22 nt, Homo sapiens
ugcaacuuaccugagucauuga

SEQ ID NO: 7 - RNA, 27 nt, Homo sapiens
auugaucaucgacacuucgaacgcaau

SEQ ID NO: 8 - RNA, 22 nt, Homo sapiens
uugggaucauuuugcauccaua

SEQ ID NO: 9 - RNA, 23 nt, Homo sapiens
uucauuugguauaaaccgcgauu

SEQ ID NO: 10 - RNA, 22 nt, Homo sapiens
caacuagacugugagcuucuag

SEQ ID NO: 11 - RNA, 21 nt, Homo sapiens
uaaggcacccuucugaguaga

SEQ ID NO: 12 - RNA, 21 nt, Homo sapiens
agagaagaagaucagccugca

SEQ ID NO: 13 - RNA, 22 nt, Homo sapiens
uaacacugucugguaacgaugu

SEQ ID NO: 14 - RNA, 21 nt, Homo sapiens
gcaguccaugggcauauacac

SEQ ID NO: 15 - RNA, 22 nt, Homo sapiens
cugccaauuccauaggucacag

SEQ ID NO: 16 - RNA, 22 nt, Homo sapiens
agaggcuggccgugaugaauuc

SEQ ID NO: 17 - RNA, 21 nt, Homo sapiens
uugaaaggcuauuucuugguc

SEQ ID NO: 18 - RNA, 23 nt, Homo sapiens
ucucuggagggaagcacuuucug

SEQ ID NO: 19 - RNA, 22 nt, Homo sapiens
caagaaccucaguugcuuuugu

SEQ ID NO: 20 - RNA, 22 nt, Homo sapiens
cgucaacacuugcugguuuccu

SEQ ID NO: 21 - RNA, 21 nt, Homo sapiens
uaaggcacccuucugaguaga

SEQ ID NO: 22 - RNA, 19 nt, Homo sapiens
agcugucugaaaaugucuu

SEQ ID NO: 23 - RNA, 20 nt, Homo sapiens
guccgcucggcgguggccca

SEQ ID NO: 24 - RNA, 23 nt, Homo sapiens
aggaagcccuggaggggcuggag

SEQ ID NO: 25 - RNA, 21 nt, Homo sapiens
agcuacaucuggcuacugggu

SEQ ID NO: 26 - RNA, 22 nt, Homo sapiens
auaaagcuagauaaccgaaagu